(19) United States
(12) Patent Application Publication
(10) Pub. No.: US 2015/0351701 A1
(43) Pub. Date: Dec. 10, 2015

(54) Methods, Computer-Readable Media, and Systems for Predicting, Detecting the Onset of, and Preventing a Seizure

(71) Applicants: Karen A. Moxon, Collingswood, NJ (US); Amrit Misra, Plainsboro, NJ (US)

(72) Inventors: Karen A. Moxon, Collingswood, NJ (US); Amrit Misra, Plainsboro, NJ (US)

(73) Assignee: DREXEL UNIVERSITY, Philadelphia, PA (US)

(21) Appl. No.: 14/735,076

(22) Filed: Jun. 9, 2015

Related U.S. Application Data
(60) Provisional application No. 62/010,409, filed on Jun. 10, 2014.

(51) Int. Cl. A61B 5/00; A61N 1/36; A61B 5/04

(52) U.S. Cl. CPC A61B 5/7275; A61B 5/04001; A61B 5/6868; A61B 5/4094; A61N 1/3606

(57) ABSTRACT

One aspect of the invention provides a method of predicting a seizure in a subject comprising: (a) recording single neuron activity for a plurality of interneurons within the subject's mesial temporal lobe; (b) recording local field potential (LFP) within the subject's mesial temporal lobe; (c) measuring interneuron synchrony within the subject's mesial temporal lobe; and (d) detecting a pattern of interneuron activity and interneuron synchrony within the subject's mesial temporal lobe associated with an increased likelihood of a seizure. Another aspect of the invention provides a method for preventing a seizure in a subject comprising: (a) performing any one of the methods described herein; and (b) upon detection of the pattern, administering a therapeutically effective intervention to the subject to prevent onset of a seizure. Another aspect of the invention provides a non-transitory computer readable medium containing computer-readable program code including instructions for performing any of the methods described herein.

### CROSS-REFERENCE TO RELATED APPLICATION

This application claims priority under 35 U.S.C. §119(e) to U.S. Provisional Patent Application Ser. No. 62/010,409, filed Jun. 10, 2014. This application is related, but does not claim priority to, U.S. patent application Ser. No. 14/122,906, filed Nov. 27, 2013, which is the national phase of International Application No. PCT/US2012/040540, filed Jun. 1, 2012, which claims priority to U.S. Provisional Patent Application Ser. Nos. 61/568,404, filed Dec. 8, 2011, and 61/492,173, filed Jun. 1, 2011. The entire content of each of these applications is hereby incorporated by reference herein.

### BACKGROUND

With over 2 million cases in the US alone and annual expenditures topping 15 billion dollars, epilepsy represents a tangible threat to our nation's heath. Epilepsy is clinically defined as a neurological disorder consisting of repeated seizures. Seizures are, in turn, defined as a paroxysmal events characterized by abnormally excessive or synchronous neuronal activity. In general, seizures are attributed to an imbalance between inhibitory and excitatory mechanisms of the brain but the underlying pathophysiology that leads to this is still not well understood.

### SUMMARY OF THE INVENTION

In one embodiment, the present invention provides a method of predicting a seizure in a subject. The method comprises (a) recording single neuron activity for a plurality of interneurons within the subject's mesial temporal lobe; (b) recording local field potential (LFP) within the subject's mesial temporal lobe; (c) measuring interneuron synchrony within the subject's mesial temporal lobe; and (d) detecting a pattern of interneuron activity and interneuron synchrony within the subject's mesial temporal lobe associated with an increased likelihood of a seizure.

In one embodiment, the recording single neuron activity and recording local field potential steps include recordation within one or more regions of the subject's mesial temporal lobe. The region may be the subject's right hippocampus, the subject's left hippocampus, the subject's right entorhinal cortex, the subject's left entorhinal cortex, the subject's amygdala, the subject's subiculum, and the subject's septum. In another embodiment, the recording single neuron activity and recording local field potential steps include recordation within a plurality of regions of the subject's mesial temporal lobe.

In yet another embodiment, the seizure originates from a presumptive focus within a region. The region may be temporal, frontal, parietal, occipital neocortex, hippocampus, and entorhinal cortex. In another embodiment, the seizure originates from a presumptive focus within the subject's neocortex.

In one embodiment, the pattern of interneuron activity includes regional interneuron synchrony between the presumptive focus and the subject's mesial temporal lobe. In another embodiment, the pattern includes synchrony between inhibitory interneurons of the subject's mesial temporal lobe and their local field potential. In yet another embodiment, the pattern includes changes in interneuron firing rates. In one embodiment, the changes include a decrease in interneuron firing rates. In another embodiment, the changes include an increase in interneuron firing rates. In one embodiment, the change in the interneuron firing rate occurs prior to seizure onset within the mesial temporal lobe. In another embodiment, the change in the interneuron firing rate occurs more than about 20 seconds prior to seizure onset within the mesial temporal lobe. In yet another embodiment, the change in the interneuron firing rate occurs more than about 100 seconds prior to seizure onset within the mesial temporal lobe.

In one embodiment, the single neuron activity has a frequency between about 2 Hz and about 12 Hz. In another embodiment, the pattern of interneuron activity includes a relative increase in magnitude of coherence when compared to a prior time period. In another embodiment, the prior time period is a 5 minute sliding window.

In one embodiment, the detecting step includes: (a) assessing whether a probe window reaches a variance threshold relative to a background window; (b) if so, assessing whether a defined portion of a trailing window preceding the probe window also reached the variance threshold; and (c) if so, returning a positive detection.

In another aspect, the present invention provides a method for preventing a seizure in a subject. The method comprises: (a) performing any one of the methods described herein; and (b) upon detection of the pattern, administering a therapeutically effective intervention to the subject to prevent onset of a seizure. In one embodiment, the therapeutically effective intervention is an electrical stimulation. In another embodiment, the therapeutically effective intervention is the delivery of a drug.

Another aspect of the invention provides a non-transitory computer readable medium containing computer-readable program code including instructions for performing any of the methods described herein.

Another aspect of the invention provides a system. The system comprises (a) a plurality of electrodes; and (b) a processor programmed to implement any one of the methods described herein.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a diagram depicting a method 100 of predicting, detecting the onset of, and/or preventing a seizure.

FIG. 2 is a schematic depicting one exemplary approach to detecting a pattern.

FIG. 3 is a diagram depicting a system for predicting, detecting the onset of, and/or preventing a seizure.

FIGS. 4A-4C are graphs illustrating interneuron activity and interneuron field coherence prior to and after seizure onset in the MTL. In FIGS. 4A-4C, a time scale change occurs at 10 minutes prior to seizure. This is denoted in each graph by the solid vertical line on the left. FIG. 4A depicts a peri-seizure time histogram of interneuron activity aligned to seizure onset in the MTL (solid vertical line, right) and averaged over all seizures across all patients. The solid horizontal line indicates mean firing rate for a 30 minute baseline period occurring 1 hour prior to electrographic seizure onset in the MTL. Dashed horizontal lines indicate +/−3 standard deviations from baseline firing activity. Below the histogram, lines indicate a decimated rastergram of example neurons showing reduced firing rate prior to seizure. In FIGS. 4B-4C, the dashed vertical line indicates a 20 second pre-ictal window of interest where interneuron firing rate significantly decreased compared to baseline activity. There appears to be a trend towards decreasing interneuron activity in the 5 minutes prior to seizure, but this drop only reaches significance 20 seconds prior to seizure onset in MTL. Within this window, 91% of interneurons exhibit a drop in firing rate compared to baseline activity. FIG. 4B depicts an average normalized power spectrum showing no pre-ictal changes and a shift towards ictal frequencies after seizure onset. FIG. 4C depicts averaged interneuron-field coherence showing a significant elevation in coherence in the 20 seconds prior to seizure onset in MTL. This activity occurs predominantly below 11 Hz and coincides with the window of time where seizure activity has started in the neocortex but has not yet reached the MTL.

FIGS. 5A-5B are graphs illustrating pyramidal cell activity and pyramidal cell field coherence prior to and after seizure onset in the MTL. In FIGS. 5A-5B, a time scale change occurs at 10 minutes prior to seizure. This is denoted in each graph by the solid vertical line on the left. FIG. 5A depicts a peri-seizure time histogram of pyramidal cell activity aligned to seizure onset in the MTL (solid vertical line, right) and averaged over all seizures across all patients. The solid horizontal line indicates mean firing rate for a 30 minute baseline period occurring 1 hour prior to electrographic seizure onset in the MTL. The dashed horizontal lines indicate +/−3 standard deviations from baseline firing activity. Below the histogram, lines indicate a decimated rastergram of several individual neurons. No significant changes in firing rate occurred in the 5 minutes prior to seizure onset in MTL. FIG. 5B depicts averaged pyramidal cell-field coherence showing no significant change in coherence in the 20 seconds prior to seizure onset in MTL as compared to baseline, unlike interneuron-field coherence. The dashed vertical line indicates a 20-second pre-ictal window of interest where interneuron firing rate decreased significantly compared to baseline activity.

FIGS. 6A-6B are graphs illustrating information flow from seizure focus to MTL structures. FIG. 6A depicts directed field-field coherence spectrograms which show an increase in information flow from seizure focus to downstream MTL structures in the 20 seconds prior to electrographic seizure onset in the MTL. At the same time, no significant reverse information flow was observed suggesting that seizure propagation form the neocortex is associated with a unidirectional flow of ictal information. FIG. 6B depicts examples of simultaneous local field potential recordings in the seizure focus (left) and the MTL (right) that share dominant oscillation frequencies. Vertical scale bars represent 1 mV while horizontal scale bars represent 1 second.

FIGS. 7A-7C are graphs illustrating an assessment of recruitment of MTL into seizure. FIG. 7A and FIG. 7B illustrate example local field potentials of seizure event with multiunit rastergram. The vertical scale bar represents 1 mV. Lettered subplots show expanded 3 second windows of activity corresponding to horizontal bars. Both seizure events exhibit an increase in rhythmic ictal spiking and concomitant rhythmic multiunit activity as the seizure event progresses. In FIG. 7C, the top graph depicts a two second window of large amplitude ictal spiking. The middle of FIG. 7C depicts an accompanying wavelet power scalogram (0-3 kHz) and the bottom of FIG. 7C shows an expanded view of high frequency wavelet power above 400 Hz. Distinct islands of increased high frequency power are observable consistent with multiunit activity rather than the stereotypic tapered cone pattern seen with filtering artifact.

FIGS. 8A-8D illustrate techniques used in the experiments described herein. FIG. 8A depicts a post-operative MRI identifying 3 depth electrodes placed bilaterally in mesial temporal lobe. In FIG. 8B, the top graph depicts raw and high-pass filtered recordings showing clear single neuron activity. The bottom of FIG. 8B depicts two example single units in principal component space with their associated mean waveform shape. The vertical scale bar represents 250 microvolts. The horizontal scale bar represents 1 second. FIG. 8C depicts an example of simultaneous recordings at the seizure focus and in the MTL. The diamond and triangle represent clinician-determined seizure onsets in each location. Underlying unit activity in the MTL increases after seizure spreads to MTL, making the electrographic onset of seizure in the MTL a good landmark for aligning seizure events across patients. FIG. 8D depicts K-means clustering of recorded neurons showing a natural separation between putative pyramidal cells and putative interneurons.

FIG. 9 is a table illustrating a breakdown of the seven patients involved in this study by age and gender. A majority of patients exhibited onset zones in the temporal lobe and a seizure etiology involving secondary generalization. Of patients who were surgical candidates, all showed reduction in seizure frequency and severity corresponding to Engel class 1 (free of disabling seizures).

FIG. 10 is a schematic representation of an algorithm described herein. The top of FIG. 10 depicts 1-second sub-divisions breaking up a 5-minute background, 10-30 second trailing window, and 1-second detection window and their corresponding position in an ongoing pre-ictal LFP. The middle of FIG. 10 illustrates an example of simultaneous single neuron firing rate broken up in 1-second bins. The bottom of FIG. 10 illustrates an example of simultaneous unit-field coherence spectrogram from 0-16 Hz broken up into 1-second time bins. The background window is used to establish the variance of a given variable to establish a detection threshold. The trailing window reduces false detection rate by setting a requirement for a sufficient number of simultaneous supra-threshold values before returning a positive detection. The scale bar is 30 seconds, and is shared across panels.

FIGS. 11A-11C are graphs illustrating relative improvement of sensitivity false detection rate and detection latency using interneuron specific variables. FIG. 11A depicts sensitivity vs. false detection rate plots for each of the 5 variables (LFP peak-to-peak amplitude, multiunit firing rate, multiunit-field coherence, interneuron firing rate, and interneuron-field coherence) using generalized seizures having interneurons. FIG. 11B depicts a comparison of false detection rate (FDR) (left) and latency (right) across all five variables for seizures containing interneurons. A significant increase in FDR is seen for multiunit variables while significant decreases in latency are seen for interneuron variables. FIG. 11C depicts a breakdown of when detections occur relative to the seizure onsets at the focus and MTL. The scale bar represents 5 seconds and the vertical scale has been normalized to peak amplitude for display purposes. LFP variables tend to detect seizure after seizures spread to MTL while interneuron measures tend to detect seizures before they start at the focus.

FIG. 12 is a table summarizing latency, sensitivity, false detection rate, and area under the curve (AUC) values for combinations of variables (LFP amplitude, multiunit firing rate, multiunit field-coherence, interneuron firing rate, and interneuron-field coherence) using generalized seizures with interneurons, generalized seizures without interneurons, and non-generalized seizures without interneurons.

### DETAILED DESCRIPTION OF THE INVENTION

Individual seizures are typified by the development of large amplitude rhythmic oscillations, often time several orders of magnitude larger than normal background activity. The clinical presentation varies with the brain structures that are involved and can range from violent convulsions (involving basal ganglia or motor cortex) to lapses of awareness (involving specific thalamo-cortical circuits). No one underlying cause can be attributed to seizure generation, however in the case of focal epilepsy, seizures initiate from a distinct pathologic zone of onset. This zone can sometimes be associated with overt pathologies, such as mass lesions or traumatic injuries, but these are not requisite for seizure generation. From this onset zone, rhythmic ictal (seizure-related) oscillations spread across the brain and subsume normal function.

The mechanisms that allow seizures to propagate to brain regions and subsume normally functioning neural networks are not well understood and are of intense interest. Of particular interest is propagation beyond the neocortex to deeper brain regions. Cerebral blood flow studies suggest that such deeper structures like the amygdala, basal ganglia, thalamus and brainstem are activated during seizure generalization and may be, in part, responsible for the clinical manifestations of seizure. In addition to several direct routes, animal studies suggest that propagation of ictal activity to these structures may occur via mesial temporal lobe (MTL) structures like hippocampus and entorhinal cortex. Yet, despite the potentially important role of MTL structures, recruitment of MTL networks into propagating ictal activity at the neuron level has been largely unexplored. Understanding the mechanism by which this occurs is of paramount importance in order to develop more effective treatment strategies.

Therefore, to date, there remains a need for an effective method of predicting, detecting the onset of, and preventing a seizure event, particularly methods based on an understanding of seizure propagation to MTL networks. The present invention meets this need.

Aspects of the present invention provides a method of predicting a seizure in a subject, preferably a human, based on measurable patterns in interneuron activity and interneuron synchrony within the subject's mesial temporal lobe (MTL). By predicting the event before onset, the present invention may provide a warning to the patient and signal for delivery of some type of intervention that would prevent seizure onset, such as an electrical stimulus or drug delivery. In one embodiment, there are measurable decreases in interneuron firing rate, increases in interneuron coherence, and increase in regional synchrony between the seizure focus and the downstream MTL targets of propagation, upon which a prediction of a true seizure can be based. The measurable patterns, specifically interneuron coherence and interneuron firing rates, can improve seizure detection prior to the current industry standard of determining onset of a seizure.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

An “effective amount” or “therapeutically effective amount” is that amount of a compound or electrical stimulation which is sufficient to provide a beneficial effect to the subject or mammal to which the compound or electrical stimulation is administered for a therapeutic treatment. A “therapeutically effective intervention” may comprise an effective amount or therapeutically effective amount of a compound or electrical simulation.

The term “epilepsy” as used herein means a neurological disorder characterized by repeated seizures.

A “health care provider” shall be understood to mean any person providing medical care to a patient. Such persons include, but are not limited to, medical doctors (e.g., neurologists), physician's assistants, nurse practitioners (e.g., an Advanced Registered Nurse Practitioner (ARNP)), nurses, residents, interns, medical students, or the like. Although various licensure requirements may apply to one or more of the occupations listed above in various jurisdictions, the term health care provider is unencumbered for the purposes of this patent application.

The term “seizure” as used herein means a transient symptom of abnormal, excessive or synchronous neuronal activity in the brain.

A “subject” shall be understood to include any mammal including, but not limited to, humans. The term “subject” specifically includes rats. The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “spike field coherence” (SFC), or “coherence”, as used herein refers to a measure of the strength of correlation between the spike times of a neuron or neuron population and the phase of the concurrent local field potential at any given frequency as described more completely by Grasse and Moxon, J. Neurophysiol. 104, 548-58 (2010).

The term “synchrony”, as used herein means a concurrence of an event between at least two or more things in a designated timeframe. Synchrony can occur on varying temporal and spatial scales. At the cellular level, synchrony may occur between individual neurons as measured by cross-correlation or between individual neurons and the local field potential as measured by unit-field coherence.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

Within this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, and 6. This applies regardless of the breadth of the range.

### Description

Aspects of the present invention are directed towards methods and systems to predict and detect epileptic seizures. Despite extensive study, the mechanisms underlying seizure generation and propagation are poorly understood. One approach is to study changes in the neuronal activity (of inhibitory and excitatory subpopulations) that occur during the recruitment of networks into a propagating seizure to gain insight into mechanisms by which seizures spread across the brain. Recent work comparing intra- and extracellular recordings in ex-vivo preparations of human neocortex has implicated a failure in feed-forward inhibition underlying the spread of seizure. However, direct in-vivo study of inhibitory and excitatory population dynamics in the neocortex is difficult due to an inability to separate single neuron activity into excitatory and inhibitory subpopulations.

It is considerably easier to isolate these subpopulations in the mesial temporal lobe (MTL) and several studies in the rodent MTL have, indeed, demonstrated an intricate spatiotemporal interplay between inhibitory and excitatory neuron firing and their corresponding synchrony to local field potentials during the transition to seizure. While this work suggests potential mechanisms for network recruitment into seizure, no direct observations have been made in the MTL of epileptic patients.

Using single neuron recordings in the human MTL, evidence is presented that supports the hypothesis that recruitment of MTL networks into seizures of neocortical origin is preceded by specific spatiotemporal increases in synchrony. In detail, within the MTL there is a decrease in inhibitory interneuron firing that coincides with the inhibitory population becoming more coherent to their local field potentials. This increased synchrony between neurons and the local field occurs at frequencies similar to those of regional synchrony between MTL networks and the seizure focus. These results suggest a mechanism by which downstream networks are prepared for recruitment into generalizing seizures. Interestingly, these spatiotemporal changes occur prior to the first electrographic manifestation of seizure in the brain, implying that in addition to their role in seizure propagation, changes in interneuron firing and interneuron-field synchrony in the MTL may be reflective of early seizure activity in other brain structures as well, and may thus be a useful tool in developing improved early detection algorithms. The methods and systems of the present invention are based in part on the discovery that changes in interneuron firing and interneuron-field synchrony in the MTL reflect early seizure activity.

Methods of Predicting, Detecting the Onset of, and/or Preventing a Seizure

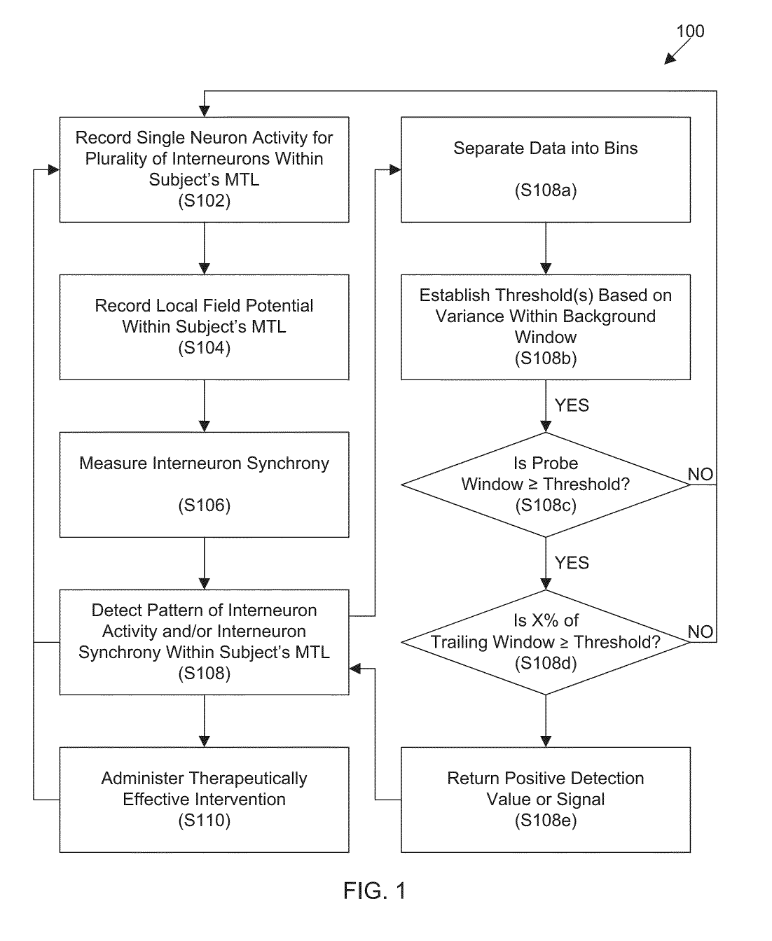

Referring now to FIG. 1, a method 100 of predicting, detecting the onset of, and/or preventing a seizure is provided. The method comprises recording single neuron activity for a plurality of interneurons within the subject's mesial temporal lobe (step S102); recording local field potential (LFP) within the subject's mesial temporal lobe (step S104); measuring interneuron synchrony within the subject's mesial temporal lobe (step S106); and detecting a pattern of interneuron activity and interneuron synchrony within the subject's mesial temporal lobe associated with an increased likelihood of a seizure (step S108).

In step S102, single neuron activity for a plurality of interneurons within the subject's mesial temporal lobe is recorded. In one embodiment, the single neuron activity includes the time of action potentials. For example, a neuron's output can be represented as 0 until it fires a spike, at which point its output becomes 1. The timing information associated with such spikes can be encoded. In some embodiments, the single neuron activity has a frequency between about 2 Hz and about 12 Hz.

In some embodiments, the recording single neuron activity and recording local field potential steps (steps S102 and S104) include recordation within one or more regions of the subject's mesial temporal lobe. The regions may include the subject's right hippocampus, the subject's left hippocampus, the subject's right entorhinal cortex, the subject's left entorhinal cortex, the subject's amygdala, the subject's subiculum, and the subject's septum. In other embodiments, the recording single neuron activity and recording local field potential steps include recordation within a plurality of regions of the subject's mesial temporal lobe.

The single neuron activity can be detected and recorded using electrodes such as those described in U.S. Pat. Nos. 6,834,200 and 8,086,316 and Karen Moxon et al., “Ceramic-based multisite electrode arrays for chronic single-neuron recording,” IEEE Trans. Biomed. Eng. 647-56 (2004) and Karen Moxon et al., “Real-time Seizure Detection System using Multiple Single Neuron Recordings,” in Proceedings of the 23rd Annual International Conference of the IEEE Engineering in Medicine and Biology Society (2001).

Consistent with current practice, electrodes can be implanted without any a priori knowledge of the location of particular interneurons. After analysis of signals from the electrode, a determination of whether a particular electrode is proximate to an interneuron can be made in accordance with conventional techniques.

In step S104, the local field potential (LFP) within the subject's mesial temporal lobe is measured. The LFP can be measured using the same electrode(s) as discussed above. LFP is a time varying voltage that can be measured by assessing the amplitude and frequency components of this time varying signal.

In step S106, interneuron synchrony within the subject's mesial temporal lobe is measured. Synchrony can be measured by analysis of spike-field coherence as discussed in D. W. Grasse & K. A. Moxon, “Correcting the Bias of Spike Field Coherence Estimators Due to a Finite Number of Spikes,” 104 J. Neurophysiology 548-58 (2010).

In step S108, a pattern of interneuron activity and/or interneuron synchrony is detected. The pattern can be associated with an increased likelihood of a seizure or the onset of a seizure. In some embodiments, the seizure originates from a presumptive focus within any of the following regions: temporal, frontal, parietal, occipital neocortex, hippocampus, and entorhinal cortex. In other embodiments, the seizure originates from a presumptive focus within the subject's neocortex.

In some embodiments, the pattern of interneuron activity includes changes in interneuron firing rates. The changes may include a decrease or an increase in interneuron firing rates. In other embodiments, the change in the interneuron firing rate may occur prior to seizure onset within the mesial temporal lobe. In some other embodiments, the change in the interneuron firing rate may occur more than about 20 seconds prior to seizure onset within the mesial temporal lobe. The change in the interneuron firing rate may also occur more than about 100 seconds prior to seizure onset within the mesial temporal lobe. In other embodiments, the pattern of interneuron activity may include a relative increase in magnitude of coherence when compared to a prior time period. The prior time period may be a 5 minute sliding window. In other embodiments, the pattern of interneuron activity may include regional interneuron synchrony between the presumptive focus and the subject's mesial temporal lobe. In some other embodiments, the pattern may include synchrony between inhibitory interneurons of the subject's mesial temporal lobe and their local field potential.

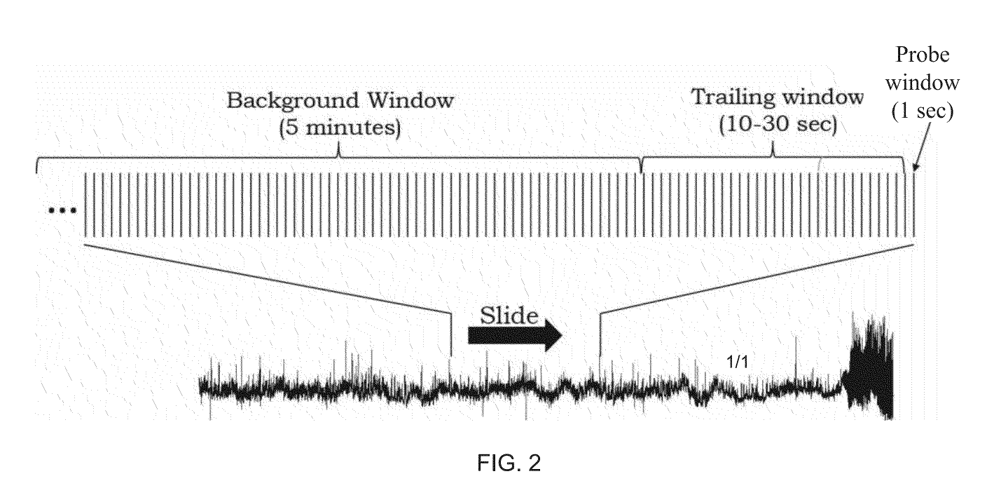

FIG. 1 and FIG. 2 graphically depict one exemplary approach to detecting a pattern. The exemplary approach comprises assessing whether a probe window reaches a variance threshold relative to a background window (steps S108a-S108c); if so, assessing whether a defined portion of a trailing window preceding the probe window also reached the variance threshold (step 108d); and if so, returning a positive detection (step 108e).

In step S108a, the measured interneuron synchrony (e.g., interneuron firing rate, i.e., the number of action potentials detected per unit of time) are separated into bins of a defined temporal duration (e.g., about 1 second). Bins can be associated with one of three windows: a background window consisting of the oldest bins (e.g., the 300 oldest bins—about 5 minutes of data), a trailing window consisting of the more recent data following the background window (e.g., between about 10 and about 30 bins—between about 10 and about 30 seconds of data), and a probe window, which will typically be of the most recent window available.

In step S108b, threshold(s) are established from variance within the background window. The variance function is defined in a variety of texts including Jan Gullberg, Mathematics: From the Birth of Numbers 976 (1997). The threshold can be set with as a multiple of (e.g., about 1, 2, 3, 4, 5, 6, 7, 8, 9, 10, and the like times) the variance. In some embodiments, the threshold is greater than the mean of the bins within the background window. The threshold (including, but not limited to the multiplier used) can be user-defined.

In step S108c, a probe window is analyzed to determine if the probe window exceeds the threshold set in step S108b.

In step S108d, if the probe window reached the threshold, the bins of the trailing window are also assessed to determine if a defined percentage (X %) of the bins in the trailing window exceeded the threshold. The value of X for each variable can be determined from interictal recordings by setting a threshold of +/−3 standard deviations from the mean and optimizing X to obtain a false detection rate of less than 1 detection per hour. This percentage can be user-defined. For example, the percentage can be between about 20% and about 100%.

In step S108e, if X % of the trailing window bins also reached the threshold, a positive detection value or signal is returned.

This method is a sliding window algorithm that can be repeated for each successive bin, with the bins constituting the background window, the trailing window, and the probe window being updated each time.

In step S110, if a pattern is detected, a therapeutically effective intervention can be administered. Suitable therapeutically effective interventions include electrical stimulation such as those produced by the CYBERONICS® VNS Therapy System and delivery of a drug. For example, the subject can receive an electrical stimulus via an implanted electrode to a particular brain region or multiple brain regions. The stimulus may be generated by a wireless signal from a wireless transceiver. Likewise, a subject may be stimulated to receive an amount of drug by a drug delivery device. For example, a drug delivery device may be implanted into the brain of the subject. In some embodiments, the drug may be directly transfused into the brain using microdialysis devices or a simple feedback alarm may be delivered to warn the subject of impending seizure so safety measures can be taken to reduce injury during seizure. In some other embodiments, the drug can be administered to the blood stream or cerebrospinal fluid (CSF) of a subject. Any drug or electrical stimulus that prevents or mitigates a predicted or detected seizure can be used with the systems and methods of the present invention.

### Interneuron Synchrony

The present invention identifies patterns of interneuron activity, where the pattern of interneuron activity includes changes in interneuron firing rates and interneuron synchrony. To identify possible motivators for the changes in interneuron firing rate, various inputs that these neurons receive were studied.

In this case, these inputs are the accumulated post-synaptic potentials that arrive from adjacent neurons. The local field potential (LFP) is effectively a summation of this cumulative activity and, so, to understand changes that occur in firing rate (neuronal output), the firing rate can be related to the local field potential (neuronal input). To measure this neuronal synchrony between firing rates and local field potentials, a measure called the unit-field coherence (Grasse and Moxon) was employed. By breaking down the local field potential into its various frequency components and assessing how synchronized output-firing is to each of these signals, insight is gained into which frequency components are most influential to the observed changes in firing rate. By then identifying generators of this this oscillatory frequency, it can be inferred which regions of the brain may be motivating the changes in firing rate that are observed.

Prior to the studies described herein, pre-ictal interneuron synchrony has not been investigated in-vivo in humans.

### Field-Field Synchrony

The concept of synchronized neural activity is central to the present understanding of epilepsy. However, synchrony can occur on varying temporal and spatial scales. At the cellular level, synchrony may occur between individual neurons as measured by cross-correlation or between individual neurons and the local field potential as measured by unit-field coherence (mentioned above). These measures occur within small volumes (<1 mm³) of tissue and when studying seizure recruitment within these volumes, these measures are made on a scale on the order of hundreds of milliseconds.

As mentioned previously, in focal epilepsy, seizures often start in a distinct focus and propagate to numerous downstream regions, so restricting an analysis to one region (such as the MTL) only provides a local view into changes associated with recruitment into seizure. To get a more comprehensive look at how seizures are spreading, it is necessary to look across brain structures, which changes the type of synchrony that must be analyzed. When looking across larger distances, i.e. between different brain regions, synchrony is generally discussed between the local field potentials at each region, and is usually measured in terms of field-field coherence or directed field-field coherence. As expected, the time scale of field-field coherence is correspondingly larger (on the order of seconds) since propagation lasts longer over greater distances. Importantly, whereas unit-field coherence provides a rough measure of which frequencies of the LFP most influence the firing of a neuronal population, directed field-field coherence is reflective of which frequencies are being used to convey information between brain structures.

To avoid confounding changes in neuronal activity associated with seizure generation with those associated with recruitment into seizure, only seizures with neocortical foci that spread to the MTL were studied. To this end, both unit-field coherence within the MTL and field-field coherence between the seizure focus and the MTL were used to complement one another and connect changes at the distant seizure focus with those in downstream MTL networks.

Implementation in Computer-Readable Media and/or Hardware

The methods described herein can be readily implemented in software that can be stored in computer-readable media for execution by a computer processor. For example, the computer-readable media can be volatile memory (e.g., random access memory and the like) and/or non-volatile memory (e.g., read-only memory, hard disks, floppy disks, magnetic tape, optical discs, paper tape, punch cards, and the like).

Additionally or alternatively, the methods described herein can be implemented in computer hardware such as an application-specific integrated circuit (ASIC).

Systems for Predicting, Detecting the Onset of, and/or Preventing a Seizure

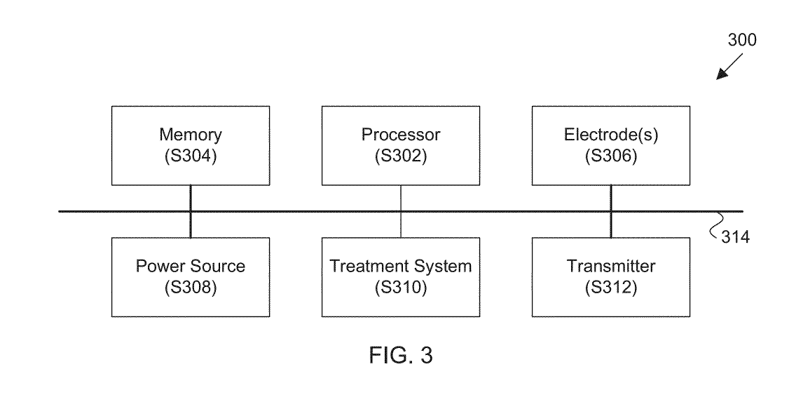

Referring now to FIG. 3, a system 300 for predicting, detecting the onset of, and/or preventing a seizure is provided. System 300 can include a processor 302. System 300 can also include memory 304 containing computer program instructions for implementing the methods described herein. Memory 304 can also be adapted and configured to store data collected from electrode 306 and/or generated by processor 302. System 300 can further include a power source 308 such as a battery, fuel cell, or the like.

System 300 can further include a treatment system 310 such as a drug delivery device (e.g., a drug pump) or an electrical stimulator.

System 300 can also include a transmitter 312 such as a wired or wireless transmitter. Transmitter 312 can enable communication of recorded and generated data to health care providers as well as tuning, configuring, and/or updating the algorithms executed by processor 302.

A system bus 314 can facilitate communication between components 302, 304, 306, 308, 310, and/or 312.

### EXPERIMENTAL EXAMPLES

The invention is further described in detail by reference to the following experimental examples. These examples are provided for purposes of illustration only, and are not intended to be limiting unless otherwise specified. Thus, the invention should be in no way construed as being limited to the following examples, but rather, should be construed to encompass any and all variations which become evident as a result of the teachings provided herein.

### Example 1

### Increased Neuronal Synchrony Prepares Mesial Temporal Networks for Recruitment into Seizures of Neocortical Origin

Despite extensive study, the mechanisms underlying seizure generalization are still poorly understood. Many efforts have focused on local propagation and the cellular mechanisms underlying the spread of ictal activity through the neocortex. Neocortical seizure activity usually propagates from the primary neocortical focus to secondary structures including the mesial temporal lobe and from there to deeper subcortical networks. Spread of ictal activity through these downstream areas and nuclei are likely responsible for altered levels of consciousness and some of the motor manifestations of seizures. Thus, the mesial temporal lobe may represent a therapeutic target for restricting propagation and mitigating the clinical impact of seizure. Unfortunately, the underlying changes in inhibitory and excitatory population firing that facilitate seizure propagation beyond the neocortex (particularly in humans) have largely gone unstudied. Described herein is evidence to support the concept that recruitment of mesial temporal networks (predominantly hippocampal) in seizures of neocortical origin is preceded by an increase in regional synchrony with the presumptive seizure focus and a decrease in inhibitory interneuron firing. Concomitantly, inhibitory populations become coherent to their local field potentials at similar frequencies as regional synchrony between mesial temporal networks and the seizure focus. These results suggest a mechanism by which downstream networks are prepared for recruitment into generalizing seizures.

Understanding the mechanisms that allow seizures to propagate to brain regions and subsume normally functioning neural networks is an area of intense study. Of particular interest is propagation beyond the neocortex to deeper brain regions. Cerebral blood flow studies suggest that such deeper structures like the amygdala, basal ganglia, thalamus and brainstem are activated during seizure generalization. In addition to several direct routes, animal studies suggest that propagation of ictal activity to these structures may occur via mesial temporal lobe (MTL) structures like hippocampus and entorhinal. Yet, despite the potentially important role of MTL structures, recruitment of MTL networks into propagating ictal activity at the neuron level has been largely unexplored. In order to investigate MTL structures as a therapeutic target for modifying the clinical presentation of generalizing seizures, an understanding of the neuronal dynamics that allow their recruitment into seizure is critical.

Applicants hypothesized that seizure propagation to MTL networks involves three components, 1) regional synchrony between the presumptive seizure focus and MTL (hippocampus, entorhinal cortex, etc.), 2) synchrony between MTL inhibitory interneurons and their local field potential (LFP), and 3) interneuron firing rate, in the time leading up to seizure. To avoid confounding seizure generation with seizure propagation, the investigation was limited to seizures with neocortical focus that secondarily generalize to MTL. By simultaneously investigating regional synchrony across the brain and interneuron-field synchrony within the MTL the frequency and time course for which 1) seizure “information” is transmitted across the brain and 2) the downstream inhibitory interneuron population is modulated were observed. By studying the overlap between these events, the likely mechanisms underlying failure in local inhibition prior to seizure propagation to the MTL were better understood.

The materials and methods employed in these experiments are now described.

### Overview

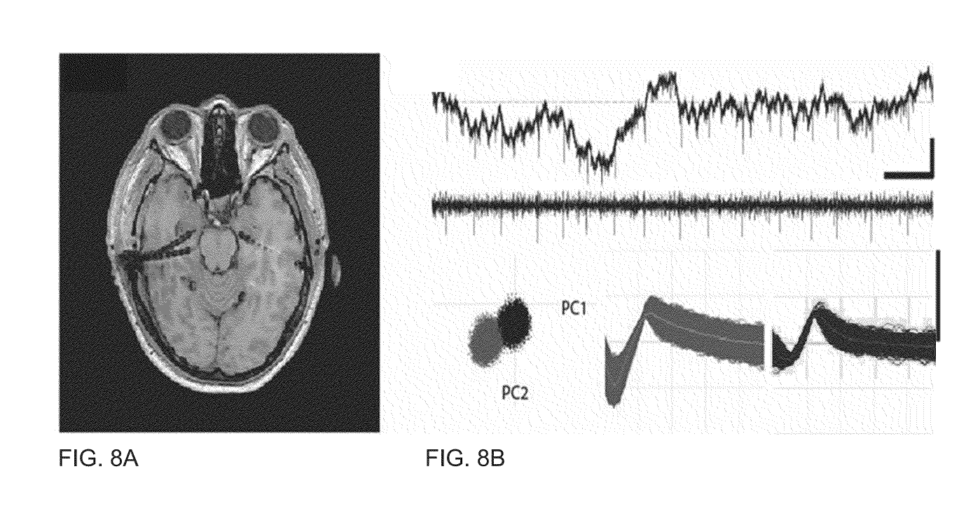

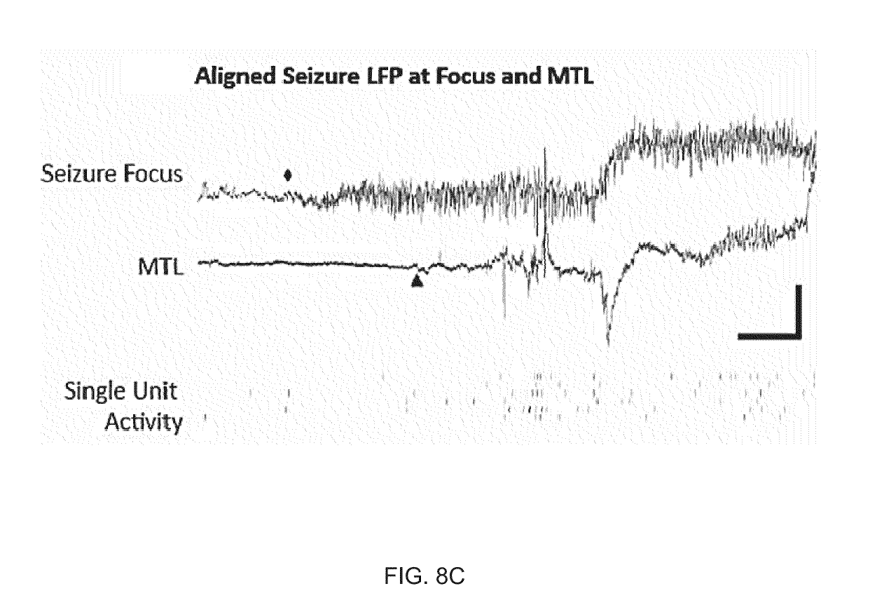

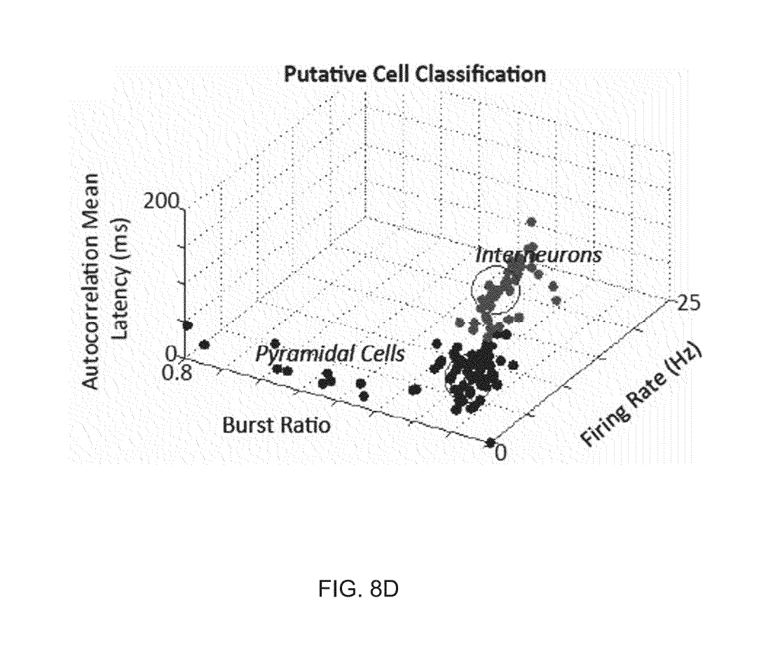

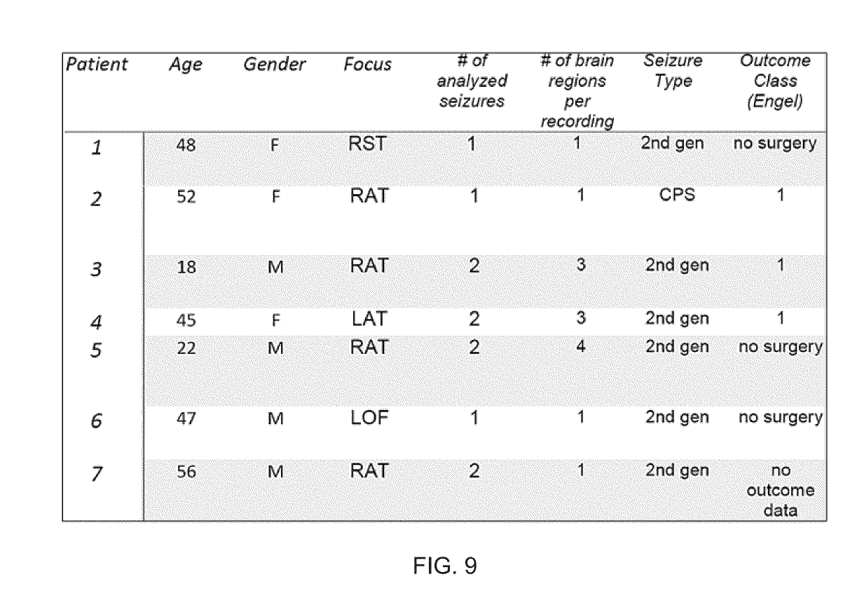

Between November of 2011 and May of 2013, seven patients undergoing diagnostic intracranial EEG (iEEG) studies for surgical resection of epileptogenic regions of the brain were implanted with modified Benkhe-Fried depth electrodes (Ad-Tech, Racine, Wis.) comprised of a platinum microwire bundle threaded through the hollowed out lumen of a standard clinical depth electrode. Implantation was performed by a board-certified neurosurgeon using established techniques from the literature (Misra et al., J. Neural Eng. 11, 026013 (2014)). Between 1 and 4 depth electrodes were placed (determined by clinical necessity) in each patient equating to between 8 and 32 microwires implanted into mesial temporal lobe (MTL) structures (Hippocampus, Entorhinal Cortex, Perirhinal Cortex, Amygdala) per patient. Electrode locations were verified by co-registered post-operative CT and pre-operative MRI38 as well as post-operative MRI (FIG. 8A). After recovery, patients were transferred to a long term monitoring unit where they were tapered off anti-epileptic medication and neural activity was continuously recorded.

### Recording and Data Collection

Neural activity was recorded (24/7) in parallel with the clinical intracranial electroencephalogram (iEEG) recordings using a wideband (0.8 Hz to 5.5 kHz) unity-gain headstage preamplifier (Neuralynx, Bozeman, Mont.). The signal was sampled by a DIGITALYNX™ high-speed data-acquisition system (Neuralynx, Bozeman, Mont.) at 32 kHz per channel providing adequate resolution for action-potential waveform discrimination. For the duration of the patient's diagnostic stay, neuronal activity was continuously recorded and time synchronized to clinical recordings.

Seizure events were identified and documented by trained technicians and clinicians in the EEG Monitoring Unit at Thomas Jefferson University Hospital. For each seizure event, seizure activity was characterized as either remaining focal or generalizing to multiple brain structures. For this study, only seizures that originated in the neocortex and subsequently generalized to MTL structures were considered. A board-certified electroencephalographer reviewed each seizure and determined the time of earliest seizure associated change in iEEG activity at the presumptive neocortical focus (global electrographic seizure onset) as well as the time of earliest seizure-associated change in the local field potential (LFP) of microwires in the MTL (MTL electrographic seizure onset).

For further analysis, neuronal activity starting 2 hours prior to global electrographic seizure onset and ending 30 minutes after seizure termination was isolated. In addition, several randomly selected 2-hour representative samples of interictal data were also isolated that was least 12 hours before and 12 hours after a seizure. Although neural signals were recorded continuously, extracellular action potential waveforms were prone to variation over time, making tracking neurons over the entire patient's stay prohibitively difficult. As such, neurons isolated from each 2 or 2.5 hour sample of data were considered independent from those identified in any other sample. For simplicity these isolated samples are henceforth referred to as independent “recordings”.

### Unit Discrimination and Classification

Single neurons were identified using a manual cluster-cutting technique as described in the literature (Chapin et al., Nat. Neurosci. 2, 664-70 (1999); Henze et al. J. Neurophysiol. 84, 390 (2000)). In brief, recorded raw signals were first high-pass filtered at 296 Hz using zero-phase finite impulse response (FIR) filter to preserve waveform shape. An amplitude threshold (usually −5 times the standard deviation of the signal) was applied to identify candidate waveforms that could be single-neuron action potentials. Waveforms that exceeded this threshold were collected and aligned. To discriminate single neurons, the first 3 principal components of the waveforms were obtained and the projection of each waveform on these component axes were used as features to cluster the data manually with OFFLINE SORTER™ software (Plexon, Dallas, Tex.) as depicted in FIG. 8D. To prevent the influence of waveform contamination during the large amplitude rhythmic LFP spiking of seizure, clustering was only done on waveforms prior to the clinician-determined electrographic onset of rhythmic LFP spiking. These waveforms were found to be stable over the course of the recording up to ˜10 seconds after clinician-determined seizure onset. Clustered units were considered true single units if (A) less than 0.5% of identified waveforms occur at inter-spike intervals that violate the refractory period (<1 msec) and (B) the autocorrelogram showed the characteristic central trough associated with a refractory period. This process was repeated for every visually approximated cluster.

Peri-ictal firing rate heterogeneity has been well documented in human epileptic patients and may, in part, be attributed to recording site variability. Because all of the recordings were in the MTL and a majority occurred in hippocampal structures, single neurons could be characterized as either putative pyramidal cell or interneuron on the basis of firing rate, autocorrelation morphology, and waveform peak-trough ratio as described in the literature (Viskontas et al. Hippocampus 17, 13 49-57 (2007)) using a custom MATLAB® k-means clustering algorithm (FIG. 8C). For this data set, the majority of recordings were hippocampal, which simplified the putative classification. Because waveform contamination interferes with single unit discrimination during the ictal period, multiunits were identified during the ictal period by band pass filtering between 400 Hz and 3000 Hz and using a 3 sigma threshold. Valid neural activity was distinguished from filtering artifacts and noise as described by Weiss et al. Brain 136(Pt 12):3796-808 (2013). In brief, raw data was downsampled to 6.5 kHz and the Morlet wavelet transform was computed. Noise and filtering artifact demonstrate a “tapered cone” morphology on the wavelet scalogram whereas physiologic neural activity demonstrate discrete “islands” of increased high-frequency power.

### Analysis of Population Firing-Rate Changes

To identify patterns in population activity for pyramidal cells and interneurons, peri-event time histograms (PETHs) were created by aligning all seizures to a common reference point (Foffani et al., J. Neurosci. Methods 135, 107-20 (2004).; Foffani et al., J. Neurosci. 24, 7266-71 (2004)). From the data, a common pattern of increased neuronal activity occurred in conjunction with the MTL electrographic seizure onset as depicted in FIG. 8B making this a reasonable alignment point across seizures. Each neuron's firing rate was then normalized to its average firing rate during the baseline period (1 hour to 30 minutes prior to seizure) to identify influences of seizure propagation within the 30 minutes prior to seizure onset on neuronal activity. The normalized firing rate was binned in 2 second intervals. For interneurons and pyramidal cells separately, these binned histograms were then averaged over all cells and all seizures. Activity from the baseline period was used to establish significance thresholds for changes around onset (e.g., mean +/−3 standard deviations from baseline firing rate). The selection of baseline was validated as described in the section “Baseline and Interictal Periods” section herein and was shown to be not different from average interictal activity. By averaging across neurons and across seizures when aligned to MTL electrographic seizure onset, the neuronal activity is time-locked to the MTL seizure onset and common firing-rate changes of the different cell populations become evident. A pre-ictal window of interest showing significant change in population activity was identified and further analyzed for changes in unit-field and field-field coherence.

### Unit-Field Coherence

To further characterize putative inhibitory and excitatory populations during the pre-ictal window of interest identified above, population average unit-field coherence was assessed for changes from baseline. Unit-field coherence was calculated using a bias-corrected approach to account for sparse firing of some neurons as discussed in Grasse and Moxon. In brief, recorded raw signal was low-pass filtered at 600 Hz offline. Filtered data was binned in 10 second intervals with 9 seconds of overlap and in 24 logarithmically spaced, non-overlapping frequency bins between 1 and 100 Hz. Unit-field coherence was calculated by first creating a spike triggered average (STA) by averaging windows of LFP around each action potential and then calculating the ratio of the average power spectrum of these segments to the power of the spike triggered average as discussed in Fries et al., J. Neurosci. 28, 4823-35 (2008).

To determine whether the unit-field coherence in the pre-ictal window of interest significantly differed from baseline periods, bins in the pre-ictal window of each frequency band were compared to an equivalent number of randomly selected bins from the same frequency bin in the baseline period by the non-parametric Mann-Whitney U test. This process was repeated 10,000 times and if the fraction of non-significant U-tests was less than 0.05, the pre-ictal window was considered significantly different at that frequency band.

### Directed Field-Field Coherence

Directed field-field coherence was calculated between the presumptive focus and the MTL using established methods (Franaszczuk et al., Electroencephalogr. Clin. Neurophysiol. 91:413-27 (1994); Kaminski et al., Biol. Cybern. 210, 203-210 (1991)) to correlate patterns of single neuron activity in the MTL with the activity at the presumptive seizure focus. In brief, raw clinical iEEG data was down-sampled from 1 kHz to 250 Hz. One clinical iEEG channel at the presumptive seizure focus and a second in proximity to microwire recordings in MTL was selected. In the case of multiple depth electrodes, one clinical MTL iEEG channel was selected to represent the local field of each microwire bundle and directed field-field coherences were calculated independently for every focus-MTL iEEG pair. The deterministic linear trend and temporal mean were then removed to satisfy stationary requirements for the subsequent analysis. The directed transfer function (DTF) described by Franaszczuk is a measure based on the concept of Granger causality and was chosen as the variable to describe directed field-field interactions. The DTF was calculated using the ECONNECTOME™ toolbox for MATLAB® software for 10 second bins with 9 seconds of overlap in both directions, using a fixed autoregressive model order of 125. The DTF was calculated for 24 logarithmically spaced frequency bands from 1 to 100 Hz as above. Statistically significant changes in coherence from baseline were identified in the same fashion as in unit-field coherence.

### Baseline and Interictal Periods

For all of the variables described above, comparisons were made between a pre-ictal window of interest and a baseline period occurring between 1 hour and 30 minutes prior to seizure onset. Single neurons could be tracked during this entire period and, therefore, neurons could be normalized to their average firing rate before averaging activity across all neurons for all seizures. To validate the selection of the baseline period and ensure activity during this period was not influenced by early, unidentified seizure activity, population averaged data during baseline periods and interictal periods were compared with respect to the following three variables: normalized firing rate, unit-field coherence, and directional field-field coherence. To assess differences in firing rate between interictal and baseline periods, a resampling technique was employed.

Differences in the population average normalized firing rate were assessed by shuffling the firing rate histogram of individual neurons and determining the 95% confidence interval of the number of significant bins (μ, +/−3σ) per hour in the resulting population average normalized firing rate histogram. This process was repeated for both baseline and interictal periods 100,000 times. For both directed field-field coherence and unit-field coherence differences between interictal and baseline periods were assessed in the same fashion. The coherence spectrograms of individual neurons were shuffled in time (not frequency) and a resultant shuffled population average spectrogram was created for baseline data and interictal data. These shuffled population averages were compared using a series of non-parametric Mann-Whitney U tests per frequency band. A false detection rate of 0.1% was used to determine significance for a given series of U tests. This process was repeated 100,000 times and the fraction of repetitions where the U test was significant was determined for each frequency band. If this fraction was greater than 0.05, baseline and interictal periods were considered to be different from each other at that frequency band.

### Ictal Periods: Multiunit Rhythmicity

MTL networks were considered recruited into a generalizing seizure if they demonstrated rhythmic multiunit firing that contributes to rhythmic LFP spiking during seizure. Rhythmic firing was determined by plotting the inter-spike interval histogram in 10 millisecond bins in a window of +/−1 second and identifying peaks in addition to the central peak that exceed a threshold of 3 times the expected value as determined by average firing rate. A multiunit was considered rhythmic if it contained one or more of these significant peaks. A seizure was considered to recruit an MTL network if one or more multiunits were recruited during a given seizure.

The results of the experiments are now described.

### Overview

In 7 patients, 11 seizures (2.5 hour recordings, each starting 2 hours prior to seizure onset) and an equivalent number of interictal periods (2 hour recordings occurring at least 12 hours from the nearest seizure event) from each patient were analyzed (FIG. 9). For each patient, neural activity was recorded from 1 to 4 MTL regions (depending upon the number of clinical depth electrodes utilized, e.g., right anterior hippocampus, left posterior hippocampus, etc.). For each seizure, data were analyzed only from regions where ictal activity propagated, as determined by a board-certified electroencephalographer. In total, 125 single neurons were recorded, 91 of which were classifiable as either putative interneuron or putative pyramidal cells based on firing rate, burst-ratio and autocorrelation morphology as described in the “Unit Discrimination and Classification” section. The remaining cells that could not be completely classified by these parameters were excluded from analysis. Prior to MTL electrographic seizure onset, putative interneurons showed a decrease in firing rate consistent with seizure spread within local neocortical regions. This breakdown of inhibition was accompanied by an increase in coherence of the interneuron activity with the local LFP in specific frequency bands known to modulate interneuron activity, while putative pyramidal cells failed to show any significant change in firing patterns. Moreover directed field-field coherence showed an increase in information flow from neocortical seizure focus to MTL at frequencies similar to the aforementioned interneuron-field coherence within the MTL.

Baseline Periods Did not Significantly Differ from Interictal Periods

For each seizure recording, a 30-minute baseline period starting 1 hour prior to the seizure was established for each recording to establish thresholds for significant changes in unit activity. To ensure that baseline periods were truly representative of interictal data for firing rate, unit-field and field-field coherence, interictal and baseline distributions were compared using a shuffling procedure described in the “Baseline and Interictal Periods” section. For average normalized firing rate, a distribution of the number of significant firing rate changes across 10,000 population average firing rate histograms with shuffled data during baseline and interictal periods was created. For both pyramidal cells and interneurons, distributions were found to be normal and the 95% confidence intervals for baseline and interictal periods completely overlap (Baseline Interneurons: 0-16 bins/hr; Interictal Interneurons: 3.5-9.5 bins/hr; Baseline Pyramidal Cells: 16-36 bins/hr; Interictal Pyramidal Cells: 23.1-32.5 bins/hr) indicating no difference in the likelihood of finding significant changes in interneuron or pyramidal cell firing between baseline and interictal periods. For directed field-field coherence in both directions (cortex→MTL and MTL→Cortex), no frequency bands were found to significantly differ between baseline and interictal periods. For interneuron-field coherence, baseline and interictal coherence distributions differed from 3-4 Hz and above 37 Hz (p<0.0004). For pyramidal cell-field coherence, baseline and interictal coherence distributions differ above 5 Hz (p<0.002). In the ensuing analysis, no pre-ictal patterns were found to occur in these frequency ranges for each respective population. As such, for frequencies of interest, baseline and interictal periods are effectively indistinguishable from one another.

### Failure in Inhibition Preceded Seizure in MTL

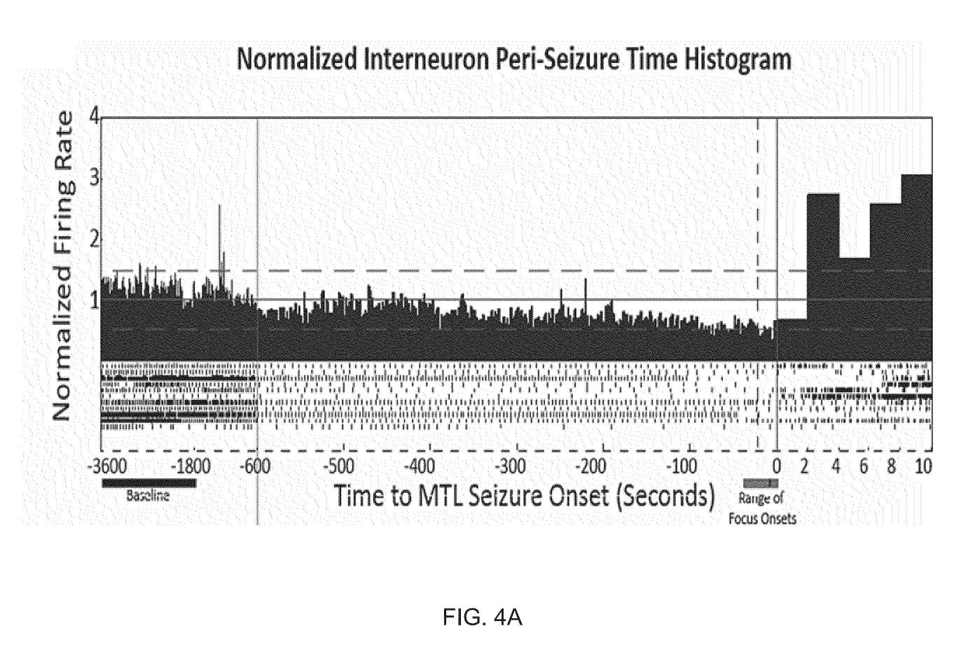

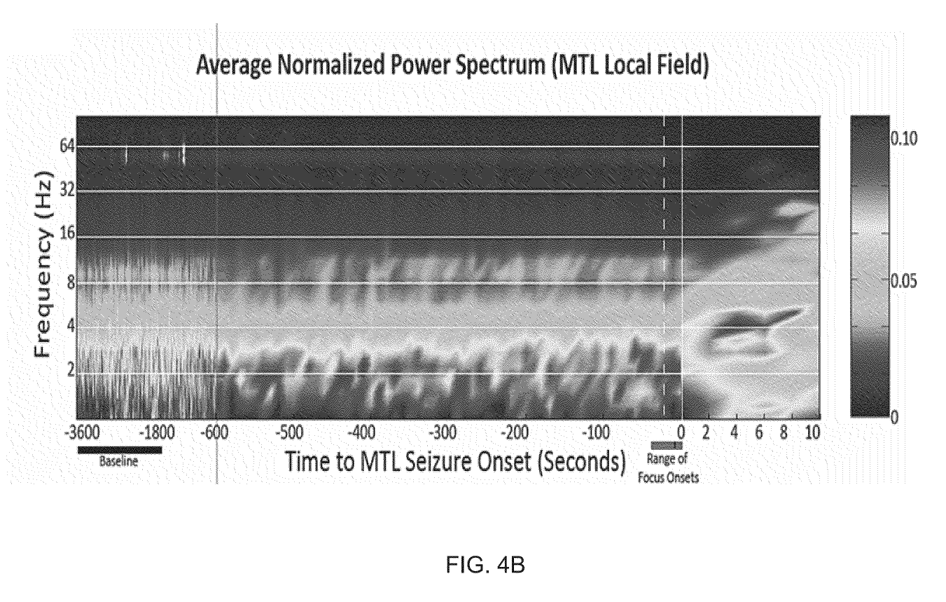

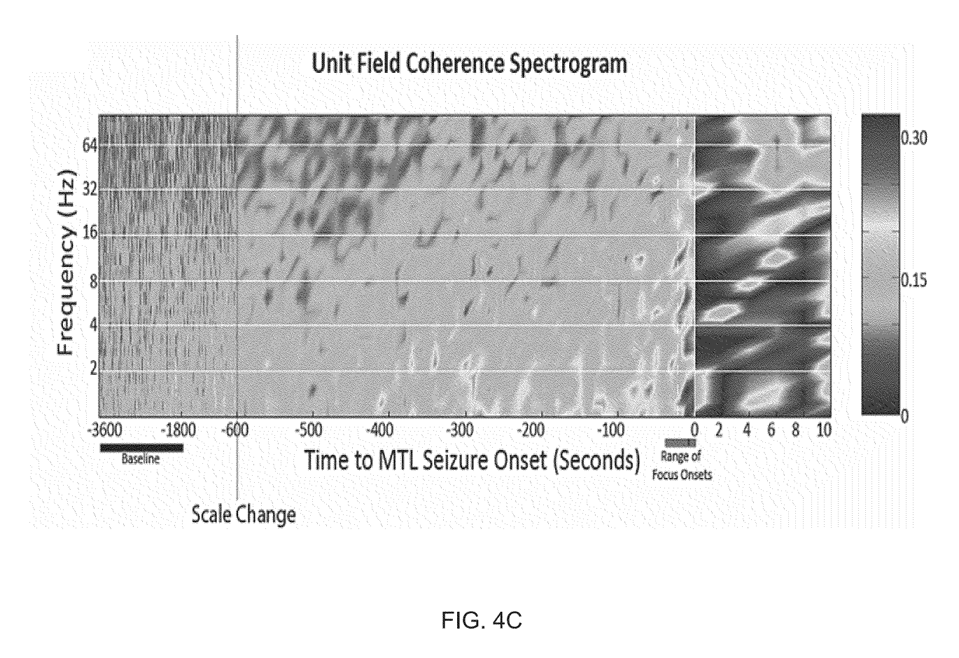

An averaged normalized firing rate histogram across all seizures for all patients was used to identify pre-ictal windows of interest containing changes in interneuron activity greater than 3 standard deviations from the mean firing rate. A significant decrease in average normalized firing rate was identified in a single window starting approximately 20 seconds prior to MTL electrographic seizure onset, when compared to baseline (FIG. 4A). This decrease was found in 91% of interneurons in all seizures with recorded interneurons (6 seizures across 5 patients). This corresponded with the period of time when the seizure started at its neocortical focus and was in the process of spreading to adjacent brain structures.

To identify possible sources that could be responsible for modulating interneuron activity during the pre-ictal window of interest, subsequent analysis characterized the unit-field coherence of this population 20 seconds prior to MTL electrographic seizure onset compared to baseline. To give context to the unit-field coherence and ensure that interpretations were physiologically relevant, an average fractional power spectrum was made to identify the dominant ongoing oscillations during the pre-ictal window of interest as depicted in FIG. 4B. As expected, the majority of the power in LFP signal was accounted for by oscillations between 1 and 8 Hz with lower frequencies being more prominent. Visual inspection of the raw data suggested that oscillations within this frequency range occurred in 2-3 second bursts.

During the 20-second pre-ictal window of interest, significant increases in interneuron-field coherence occurred at frequencies below 11 Hz (p<0.0004 for 1-3 Hz, 4-11 Hz, FIG. 4C). At the individual neuron level, 58% of interneurons originating from 5 out of the 6 seizures with interneurons across 4 out of 5 patients show increases in interneuron-field coherence that participate in the population average. Individual neurons did not, however, show a uniform increase in coherence across these three frequency ranges; rather, each neuron was associated with 1 or 2 specific frequency bands with increased interneuron-field coherence.

### Pyramidal Cells Lacked Consistent Pre-Ictal Patterns

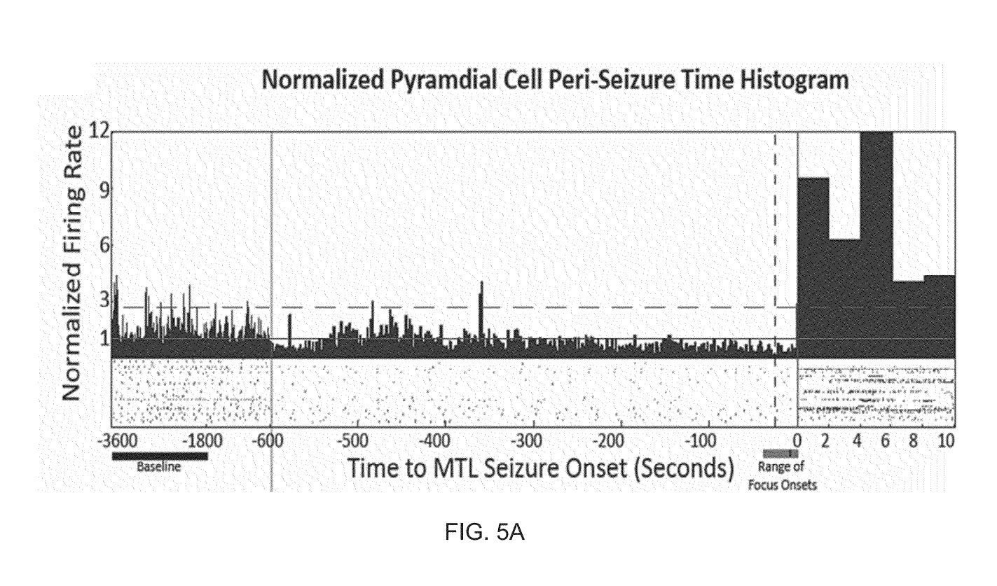

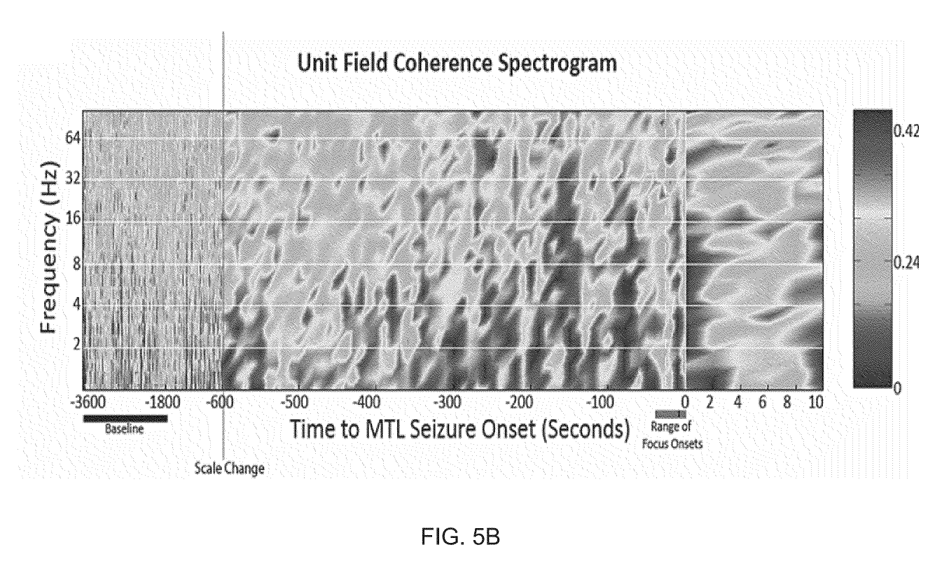

While the interneuron population showed specific pre-ictal changes in both firing rate and unit-field coherence, pyramidal cells failed to do so. The only significant change in pyramidal cell normalized firing rate occurred after MTL electrographic seizure onset (FIG. 5A) when pyramidal cells increased their firing in conjunction with the development of rhythmic ictal oscillations. Similarly, no population average pre-ictal patterns could be seen in pyramidal cell-field coherence (FIG. 5B). This result reinforces the putative classification of inhibitory and excitatory subpopulations and supports a role for inhibition in regulating seizure propagation.

Directed Field-Field Coherence Showed Cortical Influence on MTL Prior to Seizure at Frequencies that Overlapped Increases in Interneuron-Field Coherence

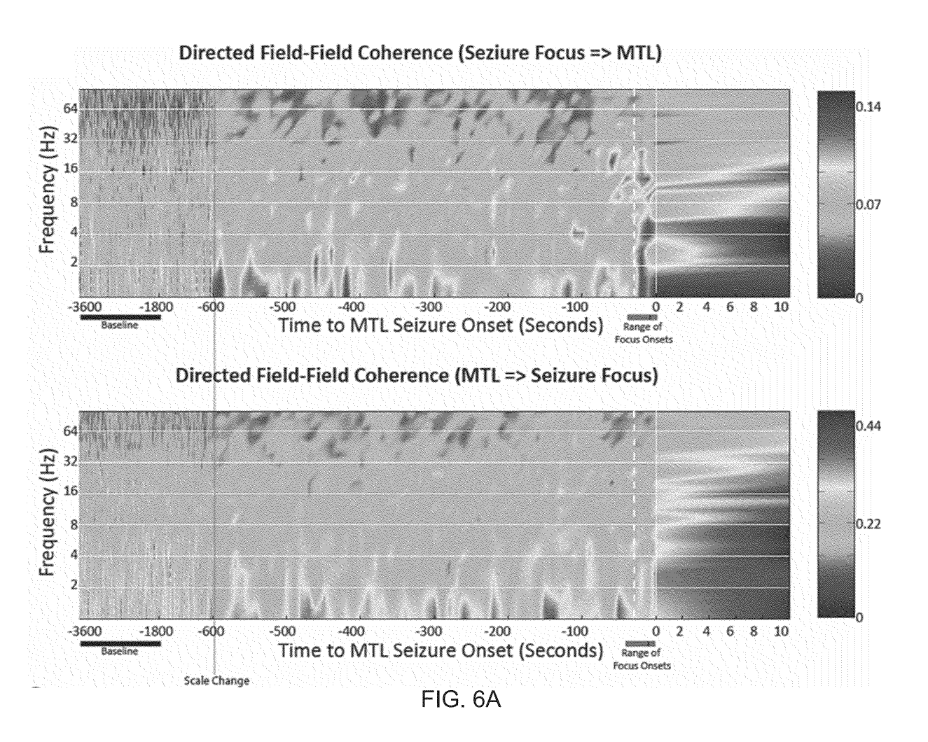

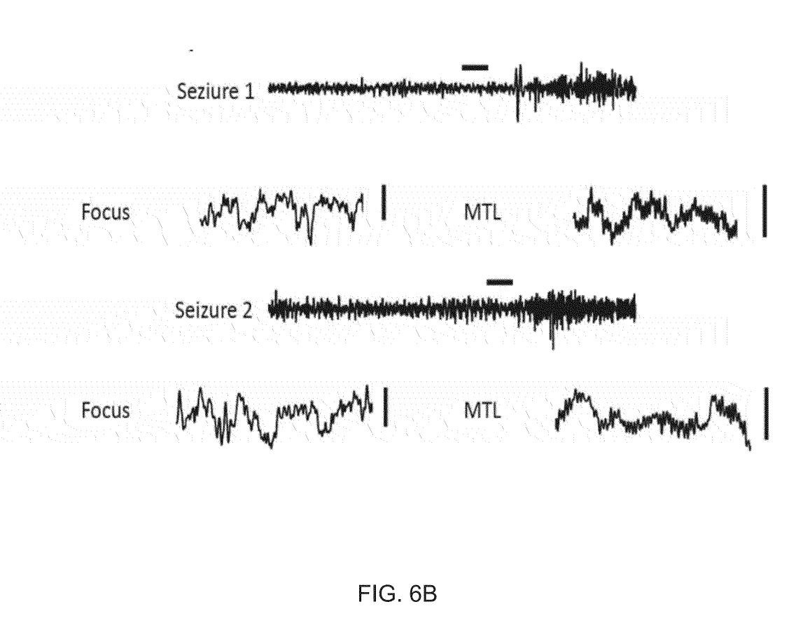

To gain insight into the influence of the neocortical seizure focus on the neuronal populations of the MTL, directed coherence analysis was performed on iEEG data from 40 pairs of clinical electrodes from the MTL and presumptive focus during 11 seizures across 7 patients. Average directional coherence across all electrode pairs in all seizures shows an increase in information flow from the presumptive focus to MTL immediately prior to MTL electrographic seizure onset at frequencies below 16 Hz (p<0.00001 for 1-2 Hz, 3-6 Hz, and 7.5-16 Hz as depicted in FIGS. 6A-6C). On a per-electrode pair basis, 65% of electrode pairs participated in this pre-ictal increase in average directional coherence. Even when restricting the analysis to only those electrode pairs from the 6 seizures with recorded interneurons, the percentage of pairs participating in the trends seen in average directional coherence remains at ˜65%.

After Seizure Onset: Assessing Recruitment of MTL into Seizure

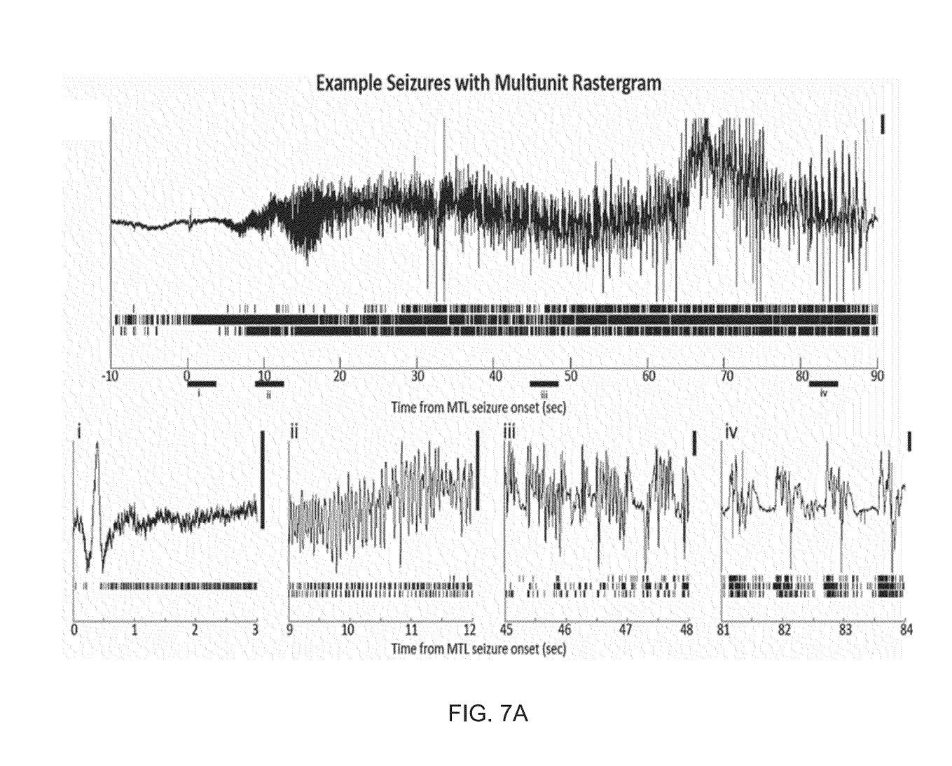

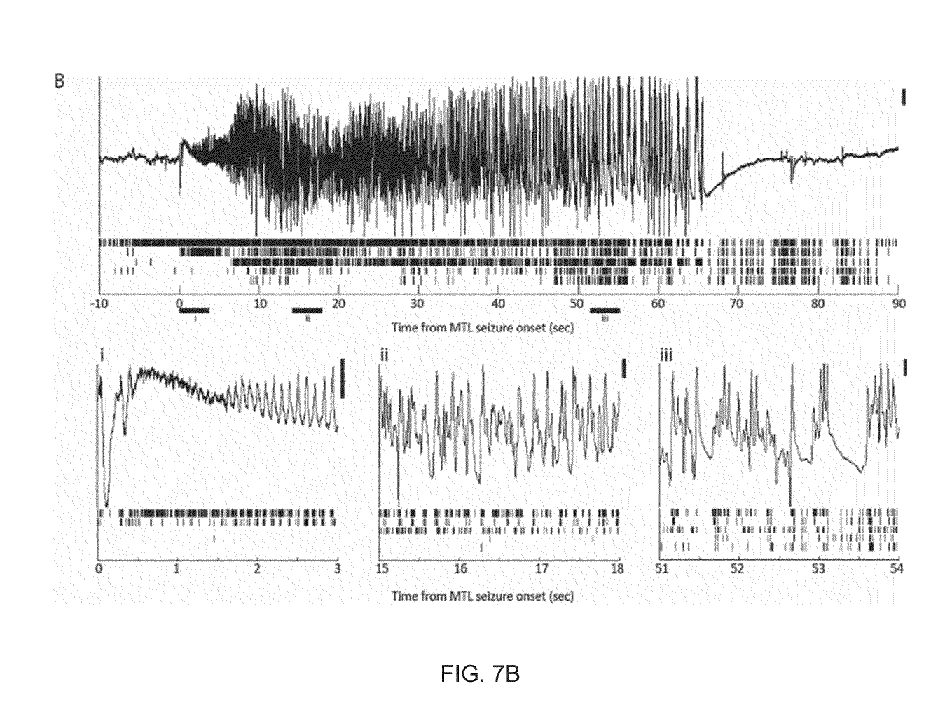

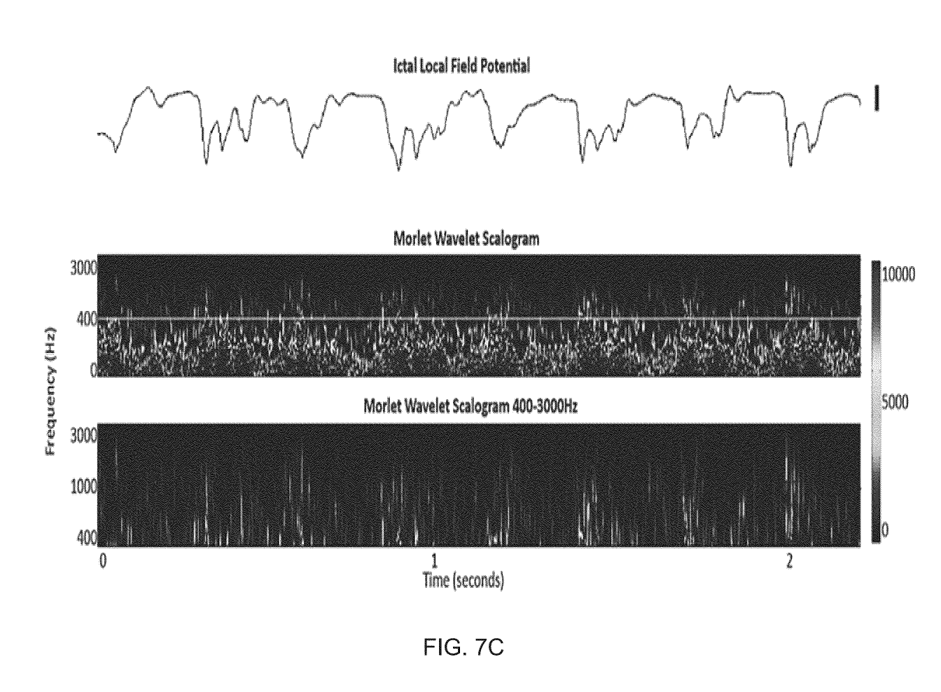

It is important to know whether neurons within the target MTL region participate in the seizure or if the seizure passes through the MTL, modulating the local field without recruiting the local neuronal populations. After MTL electrographic seizure onset, the presence of rhythmic multiunit activity at frequencies comparable to the local field was used to observe participation of the local MTL networks into the propagating seizure (FIGS. 7A-7C). Multiunits were chosen over single units as ictal spiking has been demonstrated to corrupt waveform morphology and confound single neuron template sorting. In the 11 seizures analyzed, 41 channels of multiunit activity were recorded. Multiunit activity recorded during all of the 11 seizures that were analyzed demonstrated rhythmic firing behavior. This was observed in 22 out of 24 MTL regions that exhibited ictal activity. Rhythmic multiunit behavior progressed in a series of stages (FIGS. 7A-7B). At seizure onset (i) multiunit activity was sparse or inhomogeneous, but as rhythmic ictal spiking developed in amplitude (i/ii), multiunit discharges occurred in bursts corresponding with the dominant ictal frequency. As rhythmic LFP discharges became more complex (ii/iii/iv) and involved multiple frequency components, multiunit rhythmicity became more pronounced with different multiunits rhythmically bursting in conjunction with various components of the rhythmic ictal LFP oscillation. Across all seizures studied, neurons within the MTL were found to participate in seizure activity.

### Regional Synchrony Entrained Inhibition in MTL

The results of the study described herein present evidence of a pattern of directed-field and unit-field coherence and a corresponding failure in local inhibition that precedes the propagation of ictal activity from a neocortical focus to the structures of the mesial temporal lobe. During the pre-ictal period, the distinct and overlapping frequencies of regional and interneuron-field coherence along with the notable lack of pyramidal cell-field coherence suggests that the presumptive seizure focus in the neocortex may selectively modulate downstream inhibitory subpopulations in the MTL, either directly or through intermediate networks. Moreover, the overlapping behavior of the interneuron population (i.e. simultaneous increases in interneuron-field coherence and decreases in firing rate) suggests that changes in regional synchrony may indirectly attenuate firing activity, and shut down local MTL interneuron populations.

Unlike simple field-field coherence measures, the directed field-field coherence measure allows for the assignment of a source and sink for information flow. While, this technique is particularly useful in identifying potential foci of epileptiform activity, it also provides insight into the specific frequencies that mediate ictal “information” transmission throughout the brain. Here, Applicants describe a series of frequency bands (delta-alpha) that show an increase in information flow in the seconds before MTL seizure onset as a seizure propagates from the neocortex. While the MTL and neocortex have a well-described history of mutually modulating oscillatory activity in the context of memory formation and recall, in this instance, the observed modulation is specifically associated with the propagation of seizure. More importantly, the frequencies that demonstrate directed information flow from seizure focus to MTL coincide with frequencies of interneuron-field coherence in the MTL. That the local inhibitory population becomes coherent with local field potentials (LFP) at these same frequencies is reflective of the underlying synaptic activity that is driving the MTL, the LFP being a summation of inhibitory and excitatory post-synaptic potentials that are being input to the MTL network. Thus, coherence to particular frequency components of the LFP suggests that the local MTL network is responsive to information from the upstream neocortical networks that are themselves oscillating at these frequencies (FIG. 5B). The observed overlap in frequencies of elevated directed-field coherence and interneuron-local field coherence during seizure propagation may then be interpreted as an entrainment of MTL inhibitory network activity by the seizure focus.

Pre-ictal increases in interneuron-field coherence, particularly to theta oscillations, have been described before in animal models of spontaneous seizure. Indeed, the ability of local field oscillations to selectively modulate inhibitory activity has been well documented, especially in the MTL and it is possible that similar mechanisms are at play in the context of seizure propagation. In this case, the seizure focus or upstream networks recruited into seizure may act in a similar fashion to the rodent medial septum for theta rhythms, as an extrinsic generator of oscillations that selectively entrain MTL interneuron subpopulations.

### Inhibition Failed Prior to Propagation of Seizures to MTL

In the neocortex, seizure propagation to the adjacent cortex has been described to be the result of a failure of surround inhibition to restrain the spread of ongoing seizure activity. An ictal core was identified as consisting of a population of neurons firing rhythmically with the large amplitude LFP oscillations characteristic of ictal spiking Surrounding this core, adjacent neuronal populations received excitatory synaptic barrages that created rhythmic oscillations of the LFP; however, local protective inhibition prevented these populations from firing synchronously with their oscillating LFP and, thus, the seizure failed to spread. However, if this protective inhibition failed, the rhythmic excitatory barrages entrained adjacent neuronal populations and it is suggested that this the seizure propagated to the adjacent cortex. Thus, in the neocortex, seizures are surrounded by an ictal “penumbra” that demonstrates rhythmic LFP activity without underlying rhythmic multiunit activity and represents a wave front for seizure propagation.

In this study, propagation of ictal activity to MTL shared several features with the local propagation of seizures within the neocortex. First, the observed increases in directed coherence after seizures start in the neocortex but before the MTL is recruited may be a representation of similar barrages of synaptic activity through polysynaptic circuits from the neocortex to MTL. Second, the inhibitory subpopulation of the MTL demonstrated a failure in local inhibition in a timescale that matched the increase in directed field-field coherence (excitatory barrages) and unit-field coherence but preceded the arrival of ictal activity. Finally, the failure of local inhibition in MTL was followed by an increase in rhythmic LFP spiking with concomitant rhythmic multiunit discharges after ictal activity propagated to MTL, demonstrating that the seizure was manifest in the neuronal network. Therefore, like neocortical seizure propagation, seizure propagation to MTL may also be a result of a failure of inhibitory veto.

However, the data further elaborates on the underlying mechanism that allows seizure propagation. Using unit-field coherence, it was shown that interneurons, but not pyramidal cells, exhibit increased coherence with the local MTL field preceding the arrival of ictal activity. This result suggests that the synaptic barrages from the cortex are likely to be part of a selective, feed-forward inhibitory network. Furthermore, unlike the existing neocortical seizure propagation model, prior to failure of inhibition, the interneuron population in MTL did not show a transient increase in inhibitory activity that would reflect the initial response to excitatory synaptic barrages from the seizure focus. One possibility is these neurons were not recorded. Alternatively, this may reflect potentially important differences between neocortical and MTL anatomical structure and the impact of epilepsy on those structures, e.g., synaptic remodeling or altered local inhibition.

Neocortical seizures likely spread through established circuits and enter the MTL via the mossy fiber system of the dentate gyrus. In the healthy system, these mossy fibers synapse onto both inhibitory and excitatory cells in downstream networks transmitting information from the neocortex to the MTL. Thus, in the context of receiving excitatory synaptic barrages from the neocortex, MTL pyramidal cells receive both feedforward excitation via mossy fibers and feedforward inhibition via mossy fibers synapsing onto perisomatic interneurons that maintain a balance of controlled activity within the MTL.

A hallmark of seizure-mediated reorganization in MTL is novel sprouting of mossy fibers from the dentate gyrus to downstream hippocampal networks. Simultaneously, these downstream networks exhibit a selective loss of interneurons and a reorganization of the surviving population to form new synaptic patterns onto both pyramidal cells and other surviving interneurons. However, due to aberrant rewiring of surviving MTL interneurons, local inhibitory populations of the MTL may become partially auto-inhibitory. Therefore, excessive drive from mossy fibers that entrain interneuron activity to the local field potential could slowly reduce the capacity for MTL inhibition allowing excitatory drive to dominate and seizures to propagate. This interpretation explains the observed decrease in interneuron activity without corresponding increase in pyramidal cell activity prior to propagation of seizure to MTL. Simultaneous recordings of larger numbers of interneurons are needed to determine whether the initial increase in activity was missed or if the pattern of decreasing activity is unique to the MTL.

In conclusion, this study presents evidence for a series of events immediately preceding seizure propagation to the MTL. At seizures onset, increased information from the seizure focus to downstream structures is conveyed at frequencies below 16 Hz, which is consistent with frequencies at which downstream local inhibitory populations become increasingly coherent. Concomitant with this change in coherence is a decrease in firing activity of inhibitory cells while excitatory cells show neither change in unit-field coherence or firing rate behavior. This pattern is consistent with those seen in proposed models of seizure spread within the neocortex and suggests that failure of inhibitory veto is a robust mechanism for seizure propagation. Thus, the MTL may represent a therapeutic target in terms of arresting seizure propagation to further subcortical structures like basal ganglia or septum that have been associated with the clinical presentation of seizure.

### Example 2

### Extra-Focal Changes in Inhibitory Firing and Coherence Precede the Earliest Electrographic Onset of Generalizing Seizures

The previous example identified changes in firing patterns and low-frequency unit-field coherence specific to interneurons during their recruitment into a propagating seizure. Presumably, such changes reflect and possibly facilitate the transition of a local network into a seizing state. By extension, they may also be reflective of the initial generation of seizure activity as well. Interestingly, numerous single neuron studies in epileptic patients have failed to demonstrate any such pattern prior to the first electrographic manifestation of seizure. Indeed, the activity of individual neurons in the minutes leading up to spontaneous seizures in humans has generally been described as heterogeneous. These studies have identified populations of neurons that either increase firing, decrease firing or remain unchanged during the transition to seizure. However, the underlying cause for this heterogeneity is unclear.

Of the potential causes for pre-ictal neuronal heterogeneity, the most obvious contributor is the lumping together of activity from both excitatory and inhibitory cells. Several studies suggest that inhibitory populations, in particular, change their activity during the transition to large amplitude ictal spiking. Thus isolation of such populations may draw usable information from otherwise heterogeneous patterns. Unfortunately, when studying spontaneous seizures in epileptic patients, researchers have been limited to making extracellular recordings, due to the technical and ethical complexity of intracellular recordings or other advanced electrochemical techniques to identify the presence of specific neurotransmitters in-situ. As a result, the ability to classify neurons as excitatory or inhibitory has been severely limited. Only in the last two decades have studies involving simultaneous intracellular and extracellular recordings provided guidelines for neuron classification in humans, and even this is limited to neurons recorded from mesial temporal structures. Moreover, recent studies have suggested that even if neurons can be classified as inhibitory or excitatory, they may not act with their archetypic behavior. For example, in neonatal rat seizure models of focal epilepsy, conversion of traditionally inhibitory neurons to a depolarizing (rather than hyperpolarizing) state has been observed during ictogenesis. Thus within the focus, identifying cells by patterns in their extracellular recordings alone may not be sufficient to group cells that are actively inhibiting or exciting the system.

Lastly, a large contributor to pre-ictal neuronal heterogeneity is an inherent variability between the focus of ictal activity and the location of single neuron recordings across seizures. Traditionally, studies have mitigated the effects of this variability by limiting their analyses to seizures that originate at the site of recordings (neocortical or mesial temporal). However this only increases the potential for pathologic alterations to corrupt the already limited ability to separate inhibitory from excitatory cells.

In an effort to tease out information regarding inhibition from the overall neuronal heterogeneity, the study described herein used the same population of seizures as described in the previous example (i.e., those that originate in neocortical networks and propagate to single neuron recording sites located in the mesial temporal lobe (MTL)). For this choice of seizures, the MTL facilitates separation of inhibitory and excitatory subpopulations while also having fewer confounding by pathologic changes that affect neurons within the seizure focus. Having thus reduced potential sources that may contribute to heterogeneity in single neuron activity, the resulting inhibitory subpopulation that was identified consistently provided information about impending seizure. Notably, for most of these seizures, inhibitory interneuron activity changed prior to electrographic onset of the seizure at the neocortical focus, indicating the presence of decentralized changes in network activity downstream from the seizure focus.

The materials and methods employed in these experiments are now described.

### Overview

As described in previous work (Misra et al., J. Neural Eng. 11, 026013 (2014)), high-speed recordings were performed in the mesial temporal lobe (MTL) of 7 patients undergoing diagnostic in-patient monitoring for surgical resection of epileptic brain tissue. For each patient, between 1 and 4 depth electrodes, each with an 8-wire, platinum microwire bundle, were implanted using a frameless stereotactic technique. For each depth electrode, a single microwire, not containing any single/multiunit activity, was selected as the local field potential (LFP). For the remaining wires, single neurons were extracted and clustered in principal component space using standard techniques (Lewicki, Network 9, R53-78 (1998)) with OFFLINE SORTER™ software (Plexon, Dallas, Tex.). Single units were defined as those clusters containing at least 1,000 action potentials/hour, with amplitude greater than 5 standard deviations from the mean, and fewer than 0.5% of detected action potentials occurred within 1 millisecond of one another. Each microwire recorded between 0 and 3 distinct single unit clusters. Multiunits were defined as clusters containing at least 1,000 action potentials/hour, an amplitude greater than 4.5 standard deviations from the mean and fewer than 3% of detected action potentials occurred within 1 millisecond of one another. No more than 1 multiunit was identified for each microwire. As a consequence, multiunit activity can generally be considered an aggregate of all single neuron activity recorded on a given channel.

A total of 10 seizures with neocortical foci, and ictal activity that spread to recording sites in the MTL, were captured (2 hour segments terminating in seizure). For implementation with an automated detection algorithm, each depth electrode was treated as an independent channel, resulting in a total of 19 channels of seizure data. For the purposes of assessing false detection rate, a corresponding 2 hour interictal recording (at least 12 hours from the nearest seizure) was analyzed for each channel of seizure data.

### Variable Extraction

Five variables were extracted from each channel of data in 1 second intervals: (1) LFP peak-to-peak amplitude, (2) multiunit firing rate, (3) multiunit-field coherence, (4) interneuron firing rate, and (5) interneuron-field coherence, the interneuron specific variables only being extracted for the subset of channels (7/19) that contained recorded interneurons. Firing rates were calculated by binning action potential timestamps in 1 second bins.

As described in previous work, unit-field coherences were calculated using in-house algorithms (Grasse and Moxon) in 10-second windows with 9 seconds of overlap creating a 1-second resolution to match firing rate. Unit-field coherence, at each time point, was calculated for 12 logarithmically spaced frequency bands between 1 and 16 Hz and summed to create a single coherence value for each 1-second interval.

### Algorithm Design

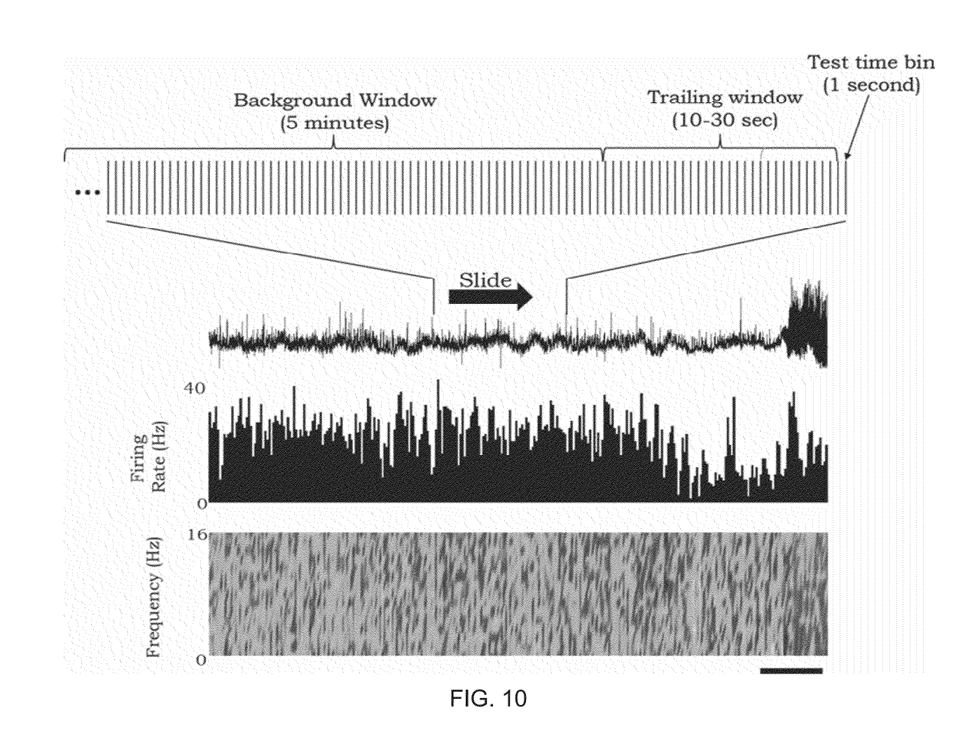

A threshold-based detection algorithm was used to detect seizures. For each variable, the value of the threshold was determined by calculating the standard deviation of a 5-minute sliding background window and applying a static multiplier. The value of this multiplier was varied between −5 and 5 to create pseudo-receiver operator characteristic curves. To reduce the effects of spurious threshold crossings on the false detection rate, the algorithm returned a positive seizure detection only if the value of the variable exceeded threshold in the current time bin, and in at least X % of a trailing window of time bins. The width of this trailing window was set to 10 seconds for local field variables and 30 seconds for single/multiunit variables. The value of X for each variable was determined from interictal recordings by setting a threshold of +/−3 standard deviations from the mean and optimizing X to obtain a false detection rate of less than 1 detection per hour. To avoid contaminating the threshold, if the algorithm returned a positive seizure detection, the background window was frozen in place for the duration of that detection and an additional 5 minutes plus the trailing window width (FIG. 10). In accordance with the average seizure duration of ˜1 minute, after a positive detection, all subsequent positive detections that occurred within 1 minute were merged into a single detection. Detections persisting longer than 1 minute were treated as multiple detections.

### Determining True and False Positives and Assessing Performance

A board-certified electroencephalographer was used to determine a set of gold-standard seizure onsets and offsets for the dataset. These times were determined at the neocortical seizure focus and not in the MTL where unit recordings and LFPs were obtained. True positives were defined as any positives detections that overlap a window of +/−60 seconds around seizure onset. This window was chosen to accommodate any variables that changed prior to the electrographic onset of seizure. For each variable, a pseudo-ROC curve was created by varying the threshold and plotting sensitivity (# of seizures detected/total number of seizures) versus false detection rate averaged over all seizures. A normalized area-under-the-curve (AUC) was calculated. The maximum sensitivity and the minimum FDR required to achieve that sensitivity was determined. For true positives, a detection latency was calculated as the difference between the start time of the merged true positive detection and the clinician defined onset of electrographic seizure at the seizure focus. The median and interquartile range for these latencies was reported.

### Statistical Comparisons

For channels where seizure activity spread to the MTL recording site, only a subset (7/19) had simultaneous interneuron recordings. To validate that this subset was representative of the whole, planned comparisons (Mann-Whitney U tests) of detection latency and false detection rate were performed for LFP amplitude, multiunit firing rate, and multiunit coherence. Having established that the subset did not differ from the whole, one-way Kruskal-Wallis tests with Mann-Whitney U tests post-hoc on the 7 seizures with interneuron activity were performed for false detection rate and detection latency to identify differences in algorithm performance when using LFP amplitude, multiunit firing rate, multiunit coherence, interneuron firing rate and interneuron coherence. For all planned comparisons a Bonferroni correction for multiple comparisons was employed.

The results of the experiments are now described.

### Overview

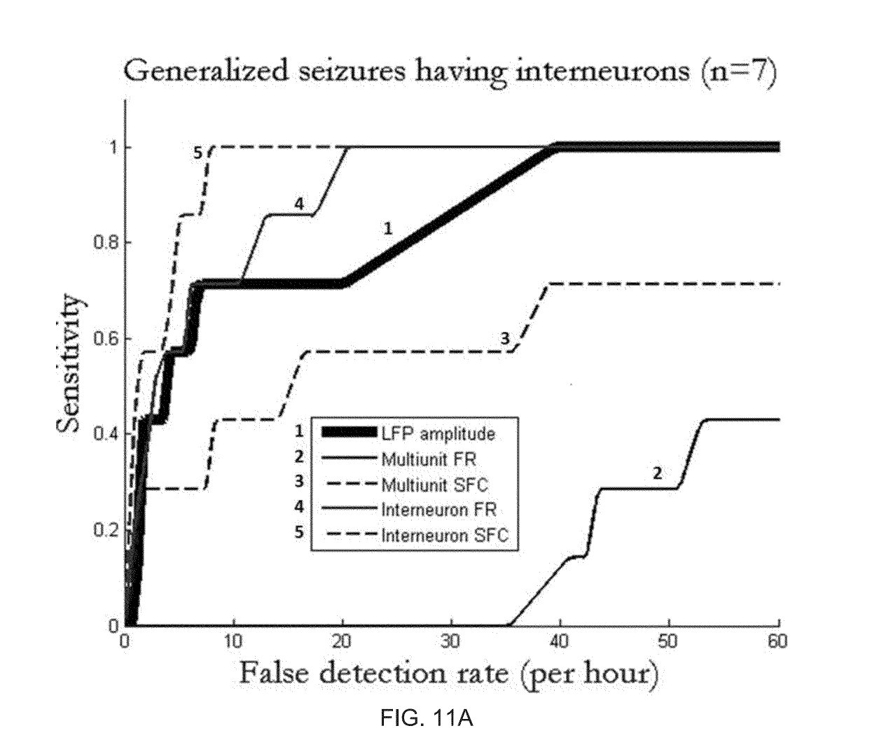

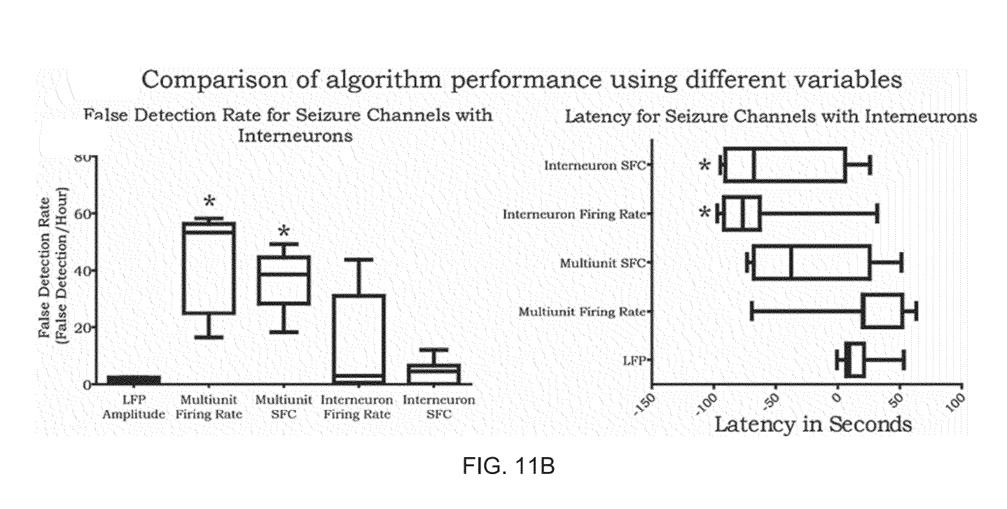

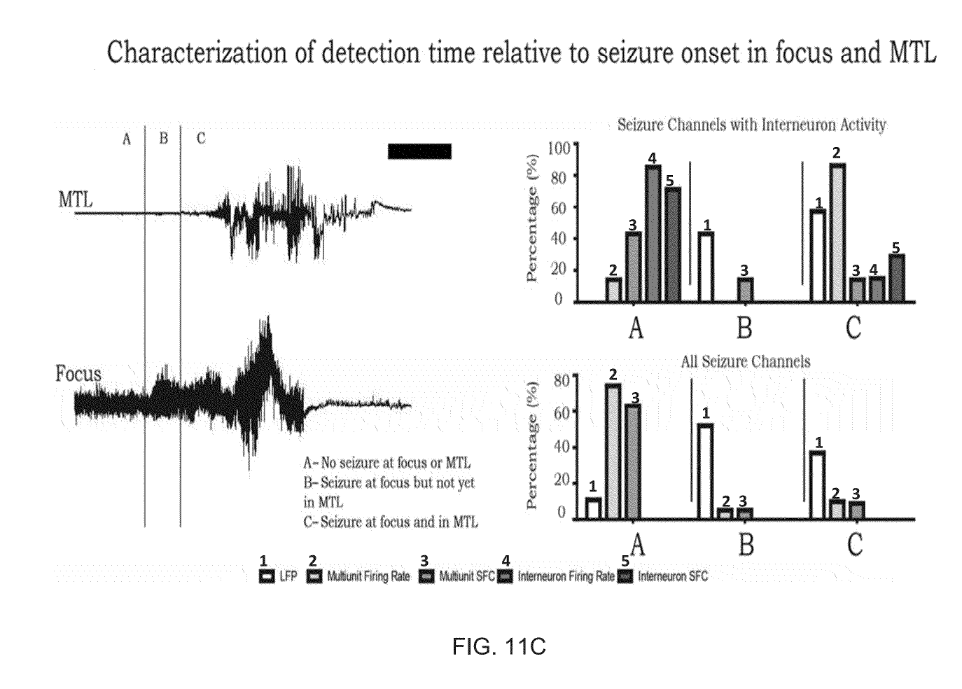

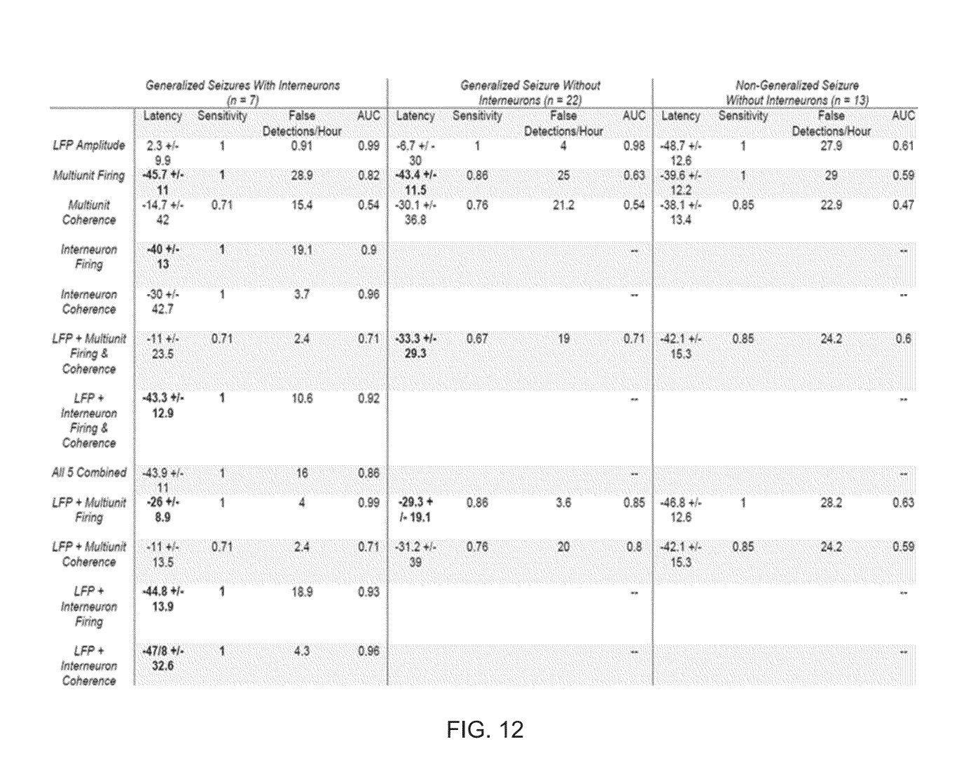

To ensure that algorithm performance across different variables was compared on an equal footing, pseudo-ROC curves (sensitivity vs. false detection rate) were created by varying the threshold as described in the methods above (FIG. 11A and FIG. 12). Sensitivities and false detection rates are reported for the threshold value that yielded the lowest false detection rate at the maximum observed sensitivity. As expected, LFP amplitude outperformed all other variables with 100% sensitivity and a median false detection rate of 1.1 detection per hour, which is comparable with published reports. Interneuron firing rate and interneuron field coherence also showed a 100% sensitivity but with an increased median FDR of 3.1 and 4.5 respectively. Interestingly, neither multiunit firing rate nor multiunit-field coherence were able to reach 100% sensitivity, and were accompanied by false detection rates of between 31 and 55 detections/hour.

Channels with Interneurons Did not Differ from Those without Interneurons

Due to the lower amplitude of interneurons in the MTL, detecting their activity using extracellular recordings is more difficult and as a result, such recordings are biased towards capturing pyramidal cells. Consequently, Applicants observed an approximate 4:1 ratio in the number of recorded pyramidal cells compared to the number of interneurons, and over half of the recordings did not have interneuron recordings at all. Algorithm performance, using LFP amplitude, multiunit firing rate and multiunit-field coherence was compared between the subset of seizure channels containing interneuron activity and the larger parent set to ensure that channels containing interneuron activity were truly representative of all seizure channels. For each of these variables, false detection rate and detection latency were compared and no significant differences were found for any of the variables (p>0.2), suggesting that while the number of channels containing interneuron are limited in this study, their findings should generalize to the larger population of seizures that propagate to the mesial temporal lobe.

### Interneurons Outperformed Multiunits in Latency to Detection and False Detection Rate

Omnibus results were significant for Kruskal-Wallis comparisons of latency (p<0.015) and false detection rate (p<0.0005) indicating that for the subset of seizures with interneuron recordings, algorithm performance significantly differed between variables (FIG. 11B, left). Post-hoc Mann-Whitney U tests show a significant decrease in the detection latency when using either interneuron firing rate or interneuron-field coherence when compared to using LFP amplitude, or multiunit firing rate, but not when using multiunit field coherence (p<0.025) as depicted in the left panel of FIG. 11B. False detection rates when using either interneuron firing rate or interneuron-field coherence did not significantly differ when compared to using LFP amplitude (p>0.2). However, using either multiunit firing rate or multiunit-field coherence resulted in a significant increase in false detection rates (p<0.03) as depicted in the right panel of FIG. 11B.

Local Field Variables Changed after Seizure Spread while Interneuron Variables Changed Prior to Focal Seizure Onset

Despite recordings occurring downstream of the neocortical seizure focus, interneuron-based variables (firing rate and interneuron-field coherence) showed an median detection latency of 68 seconds prior to focal electrographic onset of seizure compared to 9 seconds after focal electrographic seizure onset when using LFP amplitude. To account for variability in propagation time across seizures for each variable, each detection was characterized as occurring either (A) prior to electrographic seizure onset at the focus, (B) after electrographic onset at the seizure focus, but prior to electrographic onset in the MTL, or (C) after electrographic onset in the MTL. For LFP amplitude, most detection occurs during propagation or after ictal activity has spread to the MTL, whereas for interneuron variables, detections have mostly occurred prior to electrographic onset of seizure. Multiunit variables are of little interest given their high false detection rate and lower sensitivity depicted in FIG. 11C.

Data presented herein demonstrates that while heterogeneity in neuronal activity limits its usefulness in determining earliest seizure onset, isolation of the inhibitory subpopulation improves specificity, sensitivity and detection latency. Moreover, in most cases, this isolated subpopulation was found to change activity prior to the first electrographic manifestation of seizure despite not being located in the seizure focus, reinforcing the concept that seizures start prior to the first electrographic manifestation and not may be reflected outside the seizure onset zone.

### Local Field Potentials Versus Unit Activity

Local field potentials have long been the basis for seizure detection algorithms, however, successful detection relies on gross changes in the LFP such as amplitude, or dominant frequency. These changes reflect the aggregate activity of large populations of single neurons that are progressively recruited as seizures develop and propagate. As a result, changes in the local field potential are both sensitive and specific to seizure, with typical reported false detection rates at or below 1 per hour. The tradeoff is the amount of time required to recruit sufficient populations of neurons to elicit changes in the local field, which corresponds to a more prolonged latency to detection, usually on the order of seconds as is the case with the presented dataset.

In contrast, at the level of the single neuron, fluctuations in background activity are more common, making the separation of background and seizure periods more difficult. In the case of aggregate neuron activity (multiunit activity), the presented data suggest that information that is sensitive to seizure onset is lost in background fluctuations, resulting in high false detection rates and a maximum sensitivity below 100%.

The inhibitory interneuron subpopulation is an ideal target for isolation. By isolating subpopulations of inhibitory neurons from the aggregate multiunit activity, background activity becomes more consistent, resulting in fewer false detections and making it easier to find changes in activity that are specific to seizure. In this study, a decrease in interneuron firing rate and an increase in interneuron-field coherence were often associated with the seizure event.

### Downstream Interneuron Activity Changes Prior to Electrographic Seizure Onset at the Focus

While the isolation of the inhibitory subpopulation dramatically improved algorithm performance compared to aggregate multiunit activity (higher sensitivity and order of magnitude reduction in FDR) using the local field potential, the algorithm still showed a median false detection rate that was over 4 times lower than when using interneuron specific variables (e.g., firing rate and interneuron-field coherence). While this difference was not statistically significant, improvements upon the FDR may still be made by adding additional simultaneous interneurons recordings. In terms of latency, interneuron specific variables showed a significant reduction in the latency to detection, in most cases identifying a seizure event over a minute prior to first electrographic change in the LFP. This is particularly noteworthy, as the interneurons that were being recorded were in the MTL rather than in the neocortical seizure focus. This downstream change in interneuron activity could reflect either the participation of extra-focal networks in seizure generation, or more likely, downstream representations of ictogenic changes in the focus that occur prior to changes in the LFP. Prior to the electrographic onset of seizure at the focus, such clusters may be activating, but not yet engendering a change in the LFP at the seizure focus. As a result of repeated propagation to MTL during previous seizures, these clusters likely have established pathways to create downstream effects in MTL, such as the observed change in interneuron activity.

In conclusion, the study described herein has demonstrated that it is possible to tease usable information about seizure onset out of heterogeneous neuronal activity if specific subpopulations can be isolated. In the MTL, the inhibitory interneuron population lends itself to this type of isolation and has previously been identified as participating in the transition to seizure, making it an ideal candidate. By using the firing rate and unit-field coherence of MTL interneurons, seizures were reliably detected with no significant increase in FDR, as well as a statistically significant decrease in the latency to detection (compared to traditional LFP based detection). The latencies were so improved that in a majority of cases, changes in interneuron firing rate and unit-field coherence preceded the first electrographic manifestation of seizure in the seizure focus.

### EQUIVALENTS

Those skilled in the art will recognize, or be able to ascertain using no more than routine experimentation, many equivalents of the specific embodiments of the invention described herein. Such equivalents are intended to be encompassed by the following claims.

### INCORPORATION BY REFERENCE

The entire contents of all patents, published patent applications, and other references cited herein are hereby expressly incorporated herein in their entireties by reference.

1. A method of predicting a seizure in a subject, the method comprising:
(a) recording single neuron activity for a plurality of interneurons within the subject's mesial temporal lobe;
(b) recording local field potential (LFP) within the subject's mesial temporal lobe;
(c) measuring interneuron synchrony within the subject's mesial temporal lobe; and
(d) detecting a pattern of interneuron activity and interneuron synchrony within the subject's mesial temporal lobe associated with an increased likelihood of a seizure.

2. The method of claim 1, wherein the recording single neuron activity and recording local field potential steps include recordation within one or more regions of the subject's mesial temporal lobe selected from the group consisting of: the subject's right hippocampus, the subject's left hippocampus, the subject's right entorhinal cortex, the subject's left entorhinal cortex, the subject's amygdala, the subject's subiculum, and the subject's septum.

3. The method of claim 2, wherein the recording single neuron activity and recording local field potential steps include recordation within a plurality of regions of the subject's mesial temporal lobe.

4. The method of claim 1, wherein the seizure originates from a presumptive focus within a region selected from the group consisting of: temporal, frontal, parietal, occipital neocortex, hippocampus, and entorhinal cortex.

5. The method of claim 1, wherein the seizure originates from a presumptive focus within the subject's neocortex.

6. The method of claim 5, wherein the pattern includes regional interneuron synchrony between the presumptive focus and the subject's mesial temporal lobe.

7. The method of claim 5, wherein the pattern includes synchrony between inhibitory interneurons of the subject's mesial temporal lobe and their local field potential.

8. The method of claim 5, wherein the pattern includes changes in interneuron firing rates.

9. The method of claim 8, wherein the changes include a decrease in interneuron firing rates.

10. The method of claim 8, wherein the changes include an increase in interneuron firing rates.

11. The method of claim 8, wherein the change in the interneuron firing rate occurs prior to seizure onset within the mesial temporal lobe.

12. The method of claim 8, wherein the change in the interneuron firing rate occurs more than about 20 seconds prior to seizure onset within the mesial temporal lobe.

13. The method of claim 8, wherein the change in the interneuron firing rate occurs more than about 100 seconds prior to seizure onset within the mesial temporal lobe.

14. The method of claim 1, wherein the single neuron activity has a frequency between about 2 Hz and about 12 Hz.

15. The method of claim 1, wherein the pattern includes a relative increase in magnitude of coherence when compared to a prior time period.

16. The method of claim 15, wherein the prior time period is a 5 minute sliding window.

17. The method of claim 1, wherein the detecting step includes:
(a) assessing whether a probe window reaches a variance threshold relative to a background window; and
(b) if so, assessing whether a defined portion of a trailing window preceding the probe window also reached the variance threshold; and if so, returning a positive detection.

18. A method for preventing a seizure in a subject, the method comprising:
(a) performing the method of claim 1; and
(b) upon detection of the pattern, administering a therapeutically effective intervention to the subject to prevent onset of a seizure.

19. A non-transitory computer readable medium containing computer-readable program code including instructions for performing the method of claim 1.

20. A system comprising:
(a) a plurality of electrodes; and
(b) a processor programmed to implement method of claim 1.
